(19) United States
(12) Patent Application Publication
(10) Pub. No.: US 2023/0372276 A1
(43) Pub. Date: Nov. 23, 2023

(54) METHODS FOR DELIVERING MEDIUM CHAIN TRIGLYCERIDES WITH CONTROLLED PHARMACOKINETIC, SAFETY AND TOLERABILITY PROFILES

(71) Applicant: Cerecin Inc., Denver, CO (US)

(72) Inventors: Judith Anne Walker, Edinburgh (GB); Samuel T . Henderson, Golden, CO (US); Bruce H. Morimoto, Denver, CO (US)

(73) Assignee: Cerecin Inc., Denver, CO (US)

(21) Appl. No.: 18/248,186

(22) PCT Filed: Oct. 11, 2021
(86) PCT No.: PCT/US2021/054412

§ 371 Date: Apr. 6, 2023

Related U.S. Application Data
(60) Provisional application No. 63/089,797, filed on Oct. 9, 2020.

(51) Int. Cl. A61K 31/23

(52) U.S. Cl. CPC A61K 31/23

(57) ABSTRACT

The invention relates compositions of medium chain triglycerides (MCTs), and to methods for treatment with such compositions to treat conditions associated with reduced neuronal metabolism, for example Alzheimer's disease.

### CROSS REFERENCE TO RELATED APPLICATIONS

This application claims the benefit of U.S. Provisional Application No. 63/089,797, filed Oct. 9, 2020, the disclosures of which is hereby incorporated by reference in its entirety.

### FIELD OF THE INVENTION

This disclosure relates to methods for delivering pharmaceutical compositions comprising high drug loadings of medium chain triglycerides to a subject in need thereof.

### BACKGROUND OF THE INVENTION

Medium Chain Triglycerides (MCTs) are comprised of fatty acids with chain length between 5-12 carbons. MCTs have been researched extensively and have known nutritional and pharmaceutical uses. MCTs have melting points which are liquid at room temperature. Further, MCTs are relatively small and are ionizable under physiological conditions, and are generally soluble in aqueous solutions.

When intend to be used as a pharmaceutical composition, it is often desirable to achieve specific pharmacokinetic properties (e.g., Cₘₐₓ, Tₘₐₓ, etc.) based on the intended treatment.

As such, there is a need in the art for pharmaceutical compositions of MCTs that achieve specific pharmacokinetic properties.

### SUMMARY OF THE INVENTION

In an aspect, the disclosure relates to a method of administering tricaprilin for the treatment of a disease or disorder in a subject in need thereof. In certain embodiments, the method comprises administering a pharmaceutical composition comprising a therapeutically effective amount of tricaprilin to the subject in need thereof, wherein the therapeutically effective amount of tricaprilin provides a maximum serum concentration (Cₘₐₓ) of total ketones of at least 300 μmol/L. In certain embodiments, the Cₘₐₓ of total ketones at least 500 μmol/L, at least 750 μmol/L, or at least 1000 μmol/L.

In certain embodiments, the therapeutically effective amount of tricaprilin is between 30 g and 80 g per day, administered as single or divided doses.

In some embodiments, the therapeutically effective amount of tricaprilin provides a Cₘₐₓ of tricaprilin of at least 500 ng/mL.

In certain embodiments, the therapeutically effective amount of tricaprilin provides a maximum serum concentration (Cₘₐₓ) of total ketones within at least 1 hour after administration, at least 1.5 hours after administration, at least 2 hours after administration, at least 2.5 hours after administration, or at least 3 hours after administration.

In certain embodiments, the subject in need thereof is an elderly subject. In certain embodiments, the elderly subject lacks the ApoE4 genotype.

In certain embodiments, the therapeutically effective amount of tricaprilin provides a Cₘₐₓ of b-hydroxybutyrate (BHB) of at least 400 μmol/L, at least 450 μmol/L, or at least 500 μmol/L.

In certain embodiments, the therapeutically effective amount of tricaprilin provides a Cₘₐₓ of acetoacetate (AcAc) of at least 50 umol/L, at least 60 umol/L, at least 70 umol/L, at least 80 umol/L, at least 90 umol/L, or at least 100 umol/L.

In certain embodiments, the disease or disorder is a disease or disorder associated with reduced cognitive function. In certain embodiments, the disease or disorder associated with reduced cognitive function is selected from Alzheimer's Disease and Age-Associated memory impairment.

In certain embodiments, the pharmaceutical composition is formed as an emulsion for administration.

In certain embodiments, the therapeutically effective does of tricaprilin of between 30 g and 80 g per day is achieved by titrating up to the final therapeutically effective dosage. In certain embodiments, the titration is performed over 2 to 4 weeks, with adjustments in dosage of 5 g to 10 g of tricaprilin per week.

In certain embodiments, the pharmaceutical composition is administered such that no ethnicity affects in total ketone Cₘₐₓ exposure after tricaprilin administration is observed in Caucasian versus Asian subjects.

While multiple embodiments are disclosed, still other embodiments of the present disclosure will become apparent to those skilled in the art from the following detailed description, which shows and describes illustrative embodiments of the disclosure. As will be realized, the invention is capable of modifications in various aspects, all without departing from the spirit and scope of the present disclosure. Accordingly, the detailed descriptions are to be regarded as illustrative in nature and not restrictive.

### BRIEF DESCRIPTION OF THE FIGURES

FIG. 1 illustrates a graph showing various BHB concentrations for various formulations in human PK studies, in accordance with an embodiment of the disclosure.

FIG. 2 illustrates a graph showing various BHB concentrations for various formulations in rat PK studies, in accordance with an embodiment of the disclosure.

FIG. 3 illustrates a model showing AcAc Cerebral metabolic rate vs time for varying dosages of tricaprilin, in accordance with embodiments of the disclosure.

FIG. 4 illustrates a graph showing Mean (±SD) Plasma Total Ketones Concentrations over time, in accordance with embodiments of the disclosure.

FIG. 5 illustrates a graph showing Mean (±SD) Unadjusted Total Ketones Plasma Concentration-Linear Scale-Overall, in accordance with embodiments of the disclosure.

FIG. 6 illustrates a graph showing Mean (±SD) Unadjusted Tricaprilin Plasma Concentration-Linear Scale-Overall, in accordance with embodiments of the disclosure.

FIG. 7 Mean (±SD) Unadjusted Octanoic Acid Plasma Concentration-Linear Scale-Overall, in accordance with embodiments of the disclosure.

FIG. 8 illustrates a graph showing Mean Unadjusted PK Concentrations-Overall-Total Ketones (μM) (PK Population), in accordance with embodiments of the disclosure.

FIG. 9 illustrates a graph showing Mean Unadjusted PK Concentrations-Overall-Tricaprilin (ng/mL) (PK Population), in accordance with embodiments of the disclosure.

FIG. 10 illustrates a graph showing Mean Unadjusted PK Concentrations-Overall-Octanoic Acid (μM) (PK Population), in accordance with embodiments of the disclosure.

FIG. 11 illustrates a graph showing Mean plasma total ketone concentrations, in accordance with embodiments of the disclosure.

FIGS. 12A-12B show scatter plots of Cm, (FIG. 12A) and AUC₀₋ₜ (FIG. 12B) for total ketones after a single administration of 50 g AC-SD-03 (20 g tricaprilin) to healthy Chinese (n-18) or Caucasian (n=14) subjects, in accordance with an embodiment of the disclosure.

FIG. 13 illustrates the generally understood in vivo metabolism of MCTs, in accordance with an embodiment of the disclosure.

### DETAILED DESCRIPTION OF THE INVENTION

The brain is highly metabolic, so any deficiency in its metabolism results in energetic stress and ultimately in cell death. Normally, the brain relies almost exclusively on glucose as an energy substrate. The brain accounts for only 2% of body weight but, utilizes 25% of total body glucose (˜120 g /day), receives 15% of cardiac output and uses 20% of total body oxygen. As such, the body has a highly conserved physiological mechanism to utilize an alternative energy substrate in times of low glucose availability: ketone bodies.

Building on the mechanism of action of ketone bodies to act as an alternative source of fuel to brain cells which cannot metabolize glucose efficiently, the present disclosure has unexpectedly found that optimized methods of administering MCTs to provide controlled pharmacokinetic profiles and outcomes may be achieved. By way of example, optimized methods may provide controlled pharmacokinetic profiles with desired maximum (or peak) concentration (Cₘₐₓ) and desired time to reach Cₘₐₓ (Tₘₐₓ) of active agent MCTs and in vivo formation of active metabolite ketone bodies. More specifically, it was found that the pharmacokinetic profiles of MCTs and in vivo formation of active metabolite ketone bodies may be controlled. In yet other embodiments, it was found that the methods of the disclosure achieve clinical outcomes wherein no ethnicity affects in pharmacokinetic profiles (e.g., total ketone Cₘₐₓ exposure after tricaprilin administration) is observed in Caucasian versus Asian subjects.

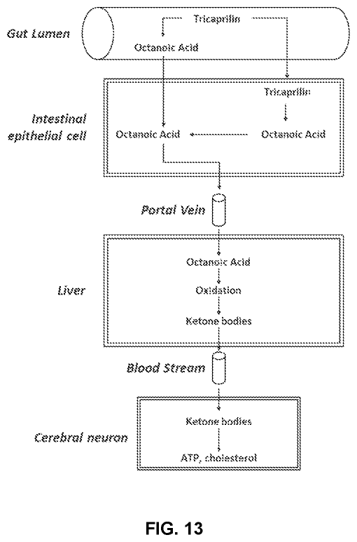

MCTs, including caprilic triglyceride or tricaprilin as described herein, are ketogenic agents, e.g., for the treatment of mild-to-moderate Alzheimer's disease (AD). However, the disclosure is not so limited, and the disclosed methods of administration may be used for the treatment of any disease, condition, or disorder that may benefit from ketogenic action. In accordance with aspects of the disclosure, tricaprilin may be administered at high doses in an attempt to compensate for regional cerebral glucose hypometabolism characteristic of AD and other diseases, conditions and disorders. Upon ingestion, tricaprilin leads to the induction of ketosis. Without intending to be limited by theory, it has been found that the formulation of the tricaprilin can influence digestion and absorption of the drug, and hence changes in formulation may influence clinical outcomes. By way of background, the in vivo metabolism of MCTs is illustrated FIG. 13.

In one embodiment, the disclosed methods of administering tricaprilin providing controlled pharmacokinetic profiles may result in elevated ketone concentrations in the body. The tricaprilin may be administered in an amount that is effective to induce hyperketonemia. In one embodiment, hyperketonemia results in ketone bodies being utilized for energy in the brain.

In one embodiment, the methods may administer tricaprilin as a pharmaceutical formulation to provide controlled circulating concentration of MCTs, e.g., tricaprilin, in the subject. The amount of circulating MCTs can be measured at a number of times post administration, and in one embodiment, is measured at a time predicted to be near the peak concentration (Cₘₐₓ) in the serum and/or plasma, but can also be measured before or after the predicted peak serum and/or plasma concentration level. Measured amounts at these off-peak times are then optionally adjusted to reflect the predicted level at the predicted peak time.

In an embodiment, the peak serum concentration (Cₘₐₓ) reached of tricaprilin or octanoic acid (OA), the MCT compound that is absorbed from the gut, is between about 350 ng/mL) to about 1500 ng/mL. In other embodiments, the peak serum concentration (Cₘₐₓ) of tricaprilin is from about 350 to about 1200 ng/mL, from about 350 to about 1000 ng/mL, from about 350 to about 950 ng/mL, etc., although variations will necessarily occur depending on the composition and subject, for example, as discussed above. In some embodiments, the peak serum concentration (Cₘₐₓ) of tricaprilin is about 400 to about 1000 ng/mL. In other embodiments, the peak serum concentration (Cₘₐₓ) of tricaprilin is at least 450 ng/mL, at least 500 ng/mL, at least 550 ng/mL, at least 600 ng/mL, at least 650 ng/mL, at least 700 ng/mL, at least 800 ng/mL, at least 850 ng/mL, at least 900 ng/mL, at least 950 ng/mL, or at least 1000 ng/mL.

In an embodiment, the time to reach Cₘₐₓ (Tₘₐₓ) of tricaprilin is about 0.5 hour to about 3 hours, e.g., about 30 minutes, about 45 minutes, about 1 hour, about 1.5 hours, about 2 hours, about 2.5 hours, or about 3 hours after administration. In another embodiment, the time to reach Cₘₐₓ (Tₘₐₓ) of MCTs is about 1 hour to about 2.5 hours. In another embodiment, the time to reach Cₘₐₓ (Tₘₐₓ) of MCTs is about 1 hour to about 2 hours. In another embodiment, the time to reach Cₘₐₓ (Tₘₐₓ) is about 0.5 hour to about 1.5 hours. In another embodiment, the time to reach Cₘₐₓ (Tₘₐₓ) of MCTs is about 0.5 hour, about 1 hour, about 1.5 hours, about 2 hours, about 2.5 hours, or about 3 hours. In another embodiment, the time to reach Cₘₐₓ (Tₘₐₓ) of MCTs is less than 3 hours, less than 2.5 hours, less than 2 hours, less than 1.5 hours, or less than 1 hour.

In an embodiment, the peak serum concentration (Cₘₐₓ) reached of total ketones is between about 350 micromole/liter (μmol/L) to about 1500 μmol/L. In other embodiments, the peak serum concentration (Cₘₐₓ) of total ketone bodies is from about 350 to about 1200 μmol/L, from about 350 to about 1000 μmol/L, from about 450 to about 1200 μmol/L, from about 500 to about 1200 μmol/L, from about 500 to about 1000 μmol/L etc., although variations will necessarily occur depending on the composition and subject, for example, as discussed above. In other embodiments, the peak serum concentration (Cₘₐₓ) of total ketone bodies is at least 450 μmol/L, at least 500 μmol/L, at least 550 μmol/L, at least 600 μmol/L, at least 650 μmol/L, at least 700 μmol/L, at least 800 μmol/L, at least μmol/L, at least 900 μmol/L, at least 950 μmol/L, or at least 1000 μmol/L.

In an embodiment, the time to reach Cₘₐₓ (Tₘₐₓ) of total ketone bodies is about 0.5 hour to about 3 hours. In another embodiment, the time to reach Cₘₐₓ (Tₘₐₓ) of total ketone bodies is about 1 hour to about 2.5 hours. In another embodiment, the time to reach Cₘₐₓ (Tₘₐₓ) of total ketone bodies is about 1 hour to about 2 hours. In another embodiment, the time to reach Cₘₐₓ (Tₘₐₓ) is about 0.5 hour to about 1.5 hours. In another embodiment, the time to reach Cₘₐₓ (Tₘₐₓ) of total ketone bodies is about 0.5 hour, about 1 hour, about 1.5 hours, about 2 hours, about 2.5 hours, or about 3 hours. In another embodiment, the time to reach Cₘₐₓ (Tₘₐₓ) of total ketone bodies is less than 3 hours, less than 2.5 hours, less than 2 hours, less than 1.5 hours, or less than 1 hour. In some embodiments, the time to reach Cₘₐₓ (Tₘₐₓ) of total ketone bodies is about 1 hour. In some embodiments, the time to reach Cₘₐₓ (Tₘₐₓ) of total ketone bodies is about 1.5 hours. In some embodiments, the time to reach Cₘₐₓ (Tₘₐₓ) of total ketone bodies is about 2 hours.

In one embodiment, the disclosed methods of administering tricaprilin may provide controlled circulating concentrations of at least one type of ketone body in the subject, including total ketone bodies, beta-hydroxybutyrate (BHB), and/or acetoacetate (AcAc). The amount of circulating ketone bodies can be measured at a number of times post administration, and in one embodiment, is measured at a time predicted to be near the peak concentration (Cₘₐₓ) in the serum and/or plasma, but can also be measured before or after the predicted peak serum and/or plasma concentration level. Measured amounts at these off-peak times are then optionally adjusted to reflect the predicted level at the predicted peak time.

In an embodiment, the peak serum concentration (Cₘₐₓ) reached of at least one ketone body (including total ketone bodies, beta-hydroxybutyrate (BHB), octanoic acid, and/or acetoacetate (AcAc)) is between about 350 micromole/liter (μmol/L) to about 1000 μmol/L. In other embodiments, the peak serum concentration (Cₘₐₓ) of at least one ketone body is from about 350 to about 950 μmol/L, from about 350 to about 900 μmol/L, from about 350 to about 850 μmol/L, from about 350 to about 800 μmol/L, from about 350 to about 750 μmol/L, from about 350 to about 700 μmol/L, from about 350 to about 650 μmol/L, from about 350 to about 550 μmol/L, from about 350 to about 500 μmol/L, or from about 350 to about 800 μmol/L, although variations will necessarily occur depending on the composition and subject, for example, as discussed above. In other embodiments, the peak serum concentration (Cₘₐₓ) of at least one ketone body is from about 400 to about 950 μmol/L, from about 400 to about 900 μmol/L, from about 400 to about 850 μmol/L, from about 400 to about 800 μmol/L, from about 400 to about 750 μmol/L, from about 400 to about 700 μmol/L, from about 400 to about 650 μmol/L, from about 400 to about 600 μmol/L, or from about 400 to about 550 μmol/L. In some embodiments, the peak serum concentration (Cₘₐₓ) of at least one ketone body is about 400 to about 600 μmol/L. In other embodiments, the peak serum concentration (Cₘₐₓ) of at least one ketone body is about 450 to about 550 μmol/L. In other embodiments, the peak serum concentration (Cₘₐₓ) of at least one ketone body is at least 350 μmol/L, at least 400 μmol/L, at least 450 μmol/L, at least 500 μmol/L at least 550 μmol/L, or at least 600 μmol/L. In other embodiments, the peak serum concentration (Cₘₐₓ) of at least one ketone body is from about 20 to about 180 μmol/L, about 20 to about 160 μmol/L, about 20 to about 140 μmol/L, about 20 to about 120 μmol/L, about 20 to about 100 μmol/L, about 20 to about 80 μmol/L, about 20 to about 60 μmol/L, or about 20 to about 40 μmol/L, although variations will necessarily occur depending on the composition and subject, for example, as discussed above.

In an embodiment, the time to reach Cₘₐₓ (Tₘₐₓ) of at least one ketone body is about 0.5 hour to about 3 hours, e.g., about 30 minutes, about 45 minutes, about 1 hour, about 1.5 hours, about 2 hours, about 2.5 hours, or about 3 hours after administration. In another embodiment, the time to reach Cₘₐₓ (Tₘₐₓ) of at least one ketone body is about 1 hour to about 2.5 hours. In another embodiment, the time to reach Cₘₐₓ (Tₘₐₓ) of at least one ketone body is about 1 hour to about 2 hours. In another embodiment, the time to reach Cₘₐₓ (Tₘₐₓ) of at least one ketone body is about 0.5 hour to about 1.5 hours. In another embodiment, the time to reach Cₘₐₓ (Tₘₐₓ) of at least one ketone body is about 0.5 hour, about 1 hour, about 1.5 hours, about 2 hours, about 2.5 hours, or about 3 hours. In another embodiment, the time to reach Cₘₐₓ (Tₘₐₓ) of at least one ketone body is less than 3 hours, less than 2.5 hours, less than 2 hours, less than 1.5 hours, or less than 1 hour. In some embodiments, the time to reach Cₘₐₓ (Tₘₐₓ) of at least one ketone body is about 1 hour. In some embodiments, the time to reach Cₘₐₓ (Tₘₐₓ) of at least one ketone body is about 1.5 hours. In some embodiments, the time to reach Cₘₐₓ (Tₘₐₓ) of at least one ketone body is about 2 hours.

In another embodiments, it was found that the methods of the disclosure achieve clinical outcomes wherein no ethnicity affects in pharmacokinetic profiles is observed in Caucasian versus Asian subjects. For example, no significant differences are observed in the tricaprilin Cₘₐₓ and Tₘₐₓ values, the total ketone Cₘₐₓ and Tₘₐₓ values, or the Cₘₐₓ and Tₘₐₓ values of ketone bodies (e.g., BHB and AcAc), following tricaprilin administration.

Described herein are several definitions. Such definitions are meant to encompass grammatical equivalents. Unless otherwise required by context, singular terms as used herein and, in the claims, shall include pluralities and plural terms shall include the singular. The use of “or” means “and/or” unless stated otherwise. Furthermore, the use of the terms “comprising,” “having,” “including,” as well as other forms, such as “includes” and “included,” are intended to be inclusive and mean that there may be additional elements other than the listed elements. Also, terms such as “element” or “component” encompass both elements and components comprising one unit and elements and components that comprise more than one subunit unless specifically stated otherwise.

As used herein, “administration” includes an in vivo use environment, such as the gastrointestinal tract, delivery by ingestion or swallowing or other such means to deliver the pharmaceutical composition, as understood by those skilled in the art. See for example, Remington: The Science and Practice of Pharmacy, 20th Edition (2000). Where the aqueous use environment is in vitro, “administration” refers to placement or delivery of the pharmaceutical composition in the in vitro test medium.

As used herein, unless otherwise specified, “% by weight” refers to “% by weight of the total composition”.

It will be appreciated by those of skill in the art, that analysis of the ketone bodies measurements/quantification can be, in some circumstances, adjusted to account for error, baseline measurements, etc. The amount of one or more ketone bodies may be determined from whole blood, plasma, serum, and or combinations thereof. The amount of one or more ketone bodies may be determined by methods known to those of skill, including, but not limited to enzymatic assays and liquid chromatography-tandem mass spectrometry (LC-MS).

Pharmaceutical compositions useful in connection with the methods of the present disclosure generally comprise a high loading of an active agent comprising at least one MCT. In accordance with certain embodiments of the disclosure, the pharmaceutical compositions of the disclosure may comprise an active agent comprising or consisting essentially of MCTs that have greater than about 95%, e.g., 98%, 99%, 99.5% or more of C8 at R₁, R₂ and R₃, and are herein referred to as caprylic triglyceride or tricaprilin (“CT”). In certain embodiments, the MCT is caprylic triglyceride or tricaprilin, as described herein. Exemplary sources of CT include MIGLYOL® 808 or NEOBEE® 895. In certain aspects, CT may be obtained from coconut or palm kernel oil, made by semi-synthetic esterification of octanoic acid to glycerin, etc.

In other embodiments, the pharmaceutical compositions may comprise an active agent comprising or consisting essentially of MCTs wherein R₁, R₂, and R₃ are fatty acids containing a six-carbon backbone (tri-C6:0). Tri-C6:0 MCT are absorbed very rapidly by the gastrointestinal tract in a number of animal model systems. The high rate of absorption results in rapid perfusion of the liver, and a potent ketogenic response. In another embodiment, the pharmaceutical compositions may comprise an active agent comprising or consisting essentially of MCTs wherein R₁, R₂, and R₃ are fatty acids containing an eight-carbon backbone (tri-C8:0). In another embodiment, the pharmaceutical compositions may comprise an active agent comprising or consisting essentially of MCTs wherein R₁, R₂, and R₃ are fatty acids containing a ten-carbon backbone (tri-C10:0). In another embodiment, the pharmaceutical compositions may comprise MCTs wherein R₁, R₂, and R₃ are a mixture of C8:0 and C10:0 fatty acids. In another embodiment, the pharmaceutical compositions may comprise an active agent comprising or consisting essentially of MCTs wherein R₁, R₂ and R₃ are a mixture of C6:0, C8:0, C10:0, and C12:0 fatty acids. In another embodiment, the pharmaceutical compositions may comprise an active agent comprising or consisting essentially of MCTs wherein greater than 95% of R₁, R₂ and R₃ are 8 carbons in length. In yet another embodiment, the pharmaceutical compositions may comprise an active agent comprising or consisting essentially of MCTs wherein the R₁, R₂, and R₃ carbon chains are 6-carbon or 10-carbon chains. In another embodiment, the pharmaceutical compositions may comprise an active agent comprising or consisting essentially of MCTs wherein about 50% of R₁, R₂ and R₃ are 8 carbons in length and about 50% of R₁, R₂ and R₃ 10 carbons in length. In one embodiment, the pharmaceutical compositions may comprise an active agent comprising or consisting essentially of MCTs wherein R₁, R₂ and R₃ are 6, 7, 8, 9, 10 or 12 carbon chain length, or mixtures thereof.

In certain embodiments, the pharmaceutical compositions may include a high drug load of an active agent comprising or consisting essentially of at least one MCT, such as tricaprilin, of at least about 30% by weight of the total composition, at least about 35% of the total composition, at least about 40% by weight of the total composition, about 30% by weight of the total composition to about 65% by weight of the total composition, about 30% by weight of the total composition to about 60% by weight of the total composition, about 35% by weight of the total composition to about 60% by weight of the total composition about 40% by weight of the total composition to about 55% by weight of the total composition, about 40% by weight of the total composition to about 50% by weight of the total composition, etc.

In certain aspects, the pharmaceutical compositions of the disclosure may comprise a high drug loading of an active agent comprising or consisting essentially of at least one MCT, at least one surfactant, and optionally an adsorbent, and/or a film forming polymer. The pharmaceutical compositions may also include a co-surfactant. In some embodiments, the pharmaceutical composition comprises at least two surfactants. In certain embodiments, the composition is a self-emulsifying, spray dried composition.

In other aspects, the at least one surfactant is selected from polyoxyl hydrogenated castor oil, polyoxyl stearate, polyoxyl hydroxystearate, lecithin, phosphatidylcholine, and combinations thereof. In certain embodiments, the solid composition comprises at least two surfactants, which may be selected from polyoxyl hydrogenated castor oil, polyoxyl stearate, polyoxyl hydroxystearate, lecithin, phosphatidylcholine, and combinations thereof. In certain embodiments, at least one of the at least two surfactants is a polyoxyl hydrogenated castor oil or polyoxyl stearate surfactant. The at least two surfactants may be present in a 2:1 to 1:1 ratio, relative to each other.

In certain aspects, the adsorbent is a silica compound, e.g., colloidal silicon dioxide (AEROSIL®, CAB-O-SIL®), amorphous silica gel (SYLOID®, SYLYSIA®), granulated silicon dioxide (AEROPERL®), silica aerogel, magnesium alumino metasilicates (NEUILIN®), calcium silicate (FLORITE®), and ordered mesoporous silicates.

In certain aspects, the film forming polymer may be polyvinylpyrrolidone (PVP), polyvinylpyrrolidone-vinyl acetate copolymer (PVP-VA), hydroxypropyl methylcellulose (HPMC), hydroxypropyl methylcellulose acetate succinate (HPMCAS), dextrans of varying molecular weights (e.g., 10000, 40000, 70000, 500000, etc.), etc. In certain embodiments, the film forming polymer is PVP or PVP-VA, in other embodiments the film forming polymer is PVP-VA.

In yet other aspects, the pharmaceutical composition of the disclosure may comprise spray dried particles having an average diameter of between about 5 μm and about 50 μm in diameter, between about 5 μm and about 30 μm in diameter, between about 5 μm and about 20 μm in diameter, between about 5 μm and about 10 μm in diameter, etc.

In other aspects, the pharmaceutical composition of the disclosure forms an emulsion in an aqueous use environment that is stable for at least about 4 hours at ambient conditions. In certain embodiments, the emulsions may have a mean droplet diameter of less than about 1000 nm, but greater than about 100 nm, e.g., between about 100 nm and 500 nm, between about 200 nm and about 300 nm, between about 160 nm and about 190 nm, etc.

In certain aspects, the tricaprilin may be administered in a pharmaceutical composition comprising a high drug loading of tricaprilin and one or more emulsion forming excipients present at a concentration sufficient to form an emulsion at room temperature. The pharmaceutical compositions may comprise the components in amounts as described herein. In some embodiments, the pharmaceutical compositions may form a stable liquid emulsion.

As described herein, the pharmaceutical compositions of the disclosure may form a liquid emulsion. An emulsion refers to a composition which, when diluted with water or other aqueous medium and gently mixed, yields a stable oil/water emulsion with a mean droplet diameter of less than about 5 μm, but greater than about 100 nm, (e.g., 0.35-1.2 μm) and which is generally polydisperse. Such an emulsion is stable, meaning there is no visibly detectable phase separation and that there is no visibly detectable crystallization.

“Gently mixed” as used herein is understood in the art to refer to the formation of an emulsion by gentle hand (or machine) mixing, such as by repeated inversions on a standard laboratory mixing machine. High shear mixing is not required to form the emulsion. Such emulsion compositions generally emulsify nearly spontaneously when introduced to an aqueous use environment.

As discussed above, the pharmaceutical compositions of the disclosure may form stable emulsions in an aqueous use environment, e.g., in water, pharmaceutically suitable aqueous solution, or when administered in vivo. By way of example, the emulsions may be stable at ambient conditions for at least about 24 hours, at least about one day, at least about 5 days, at least about 10 days, at least about one month, etc. In certain embodiments, the emulsion formed does not phase separate for the duration of stability. In certain embodiments, the emulsions may have a mean droplet diameter of less than about 5 μm, but greater than about 100 nm, (e.g., 0.35-1.2 μm).

In certain embodiments, the emulsion formed may be stable at stomach pH, e.g., at a pH of about 1 to about 3, about 1.2 to 2.9, etc. In certain embodiments, the emulsion formed may be stable at intestinal and/or colon pH, e.g., at a pH of about 5 to about 7, about 5.5 to about 6.9, etc. In certain embodiments, the emulsion formed may begin to break down or phase separate at stomach pH after about ½ to about 1 hour, but does not release the encapsulated tricaprilin until intestinal or colon pH. In this regard, without intending to be limited by theory, in-vitro digestion assays indicate that encapsulated tricaprilin is released from emulsion at intestinal and/or colon pH, which is the primary location of lipid digestion enzymes. In accordance with certain aspects of the disclosure, preferential release of tricaprilin in the intestines and/or colon rather than the stomach may increase bioavailability of the tricaprilin given the location of lipid digestion enzymes in these areas.

In certain aspects of the disclosure, the pharmaceutical compositions provide for preferential release of the high drug loading of tricaprilin in the lower gastrointestinal tract of a user. Without intending to be limited by theory, preferential release of tricaprilin in the lower gastrointestinal tract, including the colon may provide reduced stomach upset and related adverse events as compared to standard administration of non-formulated MCT oil. Further, the improved bioavailability of tricaprilin may generally lead to increased ketone body production in vivo, as compared to standard administration of non-formulated MCT oil.

In certain embodiments, the pharmaceutical compositions may include a high drug load of tricaprilin, of at least about 20% of the total composition, at least about 25% of the total composition, at least about 30% by weight of the total composition, at least about 40% by weight of the total composition, about 30% by weight of the total composition to about 65% by weight of the total composition, about 30% by weight of the total composition to about 60% by weight of the total composition, about 40% by weight of the total composition to about 50% by weight of the total composition, about 40% by weight of the total composition to about 45% by weight of the total composition, etc.

In certain aspects, the pharmaceutical compositions of the disclosure include one or more emulsion forming excipients. In certain embodiments, the one or more emulsion forming excipients may be any emulsifier capable of forming an emulsion with MCT oil. By way of example, lecithin (e.g., Phospholipon 90G), hydrogenated castor oils including Polyoxyl 40 castor oil (e.g., Kolliphor RH40), caprylate esters, sodium oleate, glycerol, citric acid esters of monoglycerides and diglycerides (e.g., Citrem), monoglycerides and diglycerides of fatty acids including Propylene Glycol Monocaprylate (e.g., Capmul PG-8), and combinations thereof. The emulsion forming excipient(s) may be present in amounts sufficient to provide desired emulsion formation. For example, in certain embodiments, the emulsion forming excipient may be present in an amount of between about 1% and about 10%, between about 1.3% and about 10%, etc., by weight of the total composition.

In certain embodiments, the emulsion forming excipients may include combinations of lecithin, Kallichore RH40, and caprylate ester emulsifiers. In other embodiments, the emulsion forming excipients may include combinations of lecithin, sodium oleate, and glycerol. In yet other embodiments, the emulsion forming excipients may include Citrem alone or in combination with monoglycerides and diglycerides of fatty acids.

In one embodiment, the pharmaceutical compositions of the disclosure are administered orally. Therapeutically effective amounts of tricaprilin can be any amount or dose sufficient to bring about the desired effect and depend, in part, on the severity and stage of the condition, the size and condition of the patient, as well as other factors readily known to those skilled in the art. The dosages can be given as a single dose, or as several doses, for example, divided over the course of several weeks, as discussed elsewhere herein.

In certain aspects, the disclosure relates to methods of treating a disease or disorder associated with reduced cognitive function in a subject in need thereof, the method comprising administering to the subject a pharmaceutical composition of the disclosure in an amount effective to elevate ketone body concentrations in said subject to thereby treat said disease or disorder. In certain embodiments, the pharmaceutical composition of the disclosure may be administered outside of the context of a ketogenic diet. For instance, in the context of the present disclosure, carbohydrates may be consumed at the same time as pharmaceutical compositions disclosed herein.

In accordance with certain aspects of the disclosure, diseases and disorders associated with reduced cognitive function including Age-Associated Memory Impairment (AAMI), Alzheimer's Disease (AD), Parkinson's Disease, Friedreich's Ataxia (FRDA), GLUT1-deficient Epilepsy, Leprechaunism, and Rabson-Mendenhall Syndrome, Coronary Arterial Bypass Graft (CABG) dementia, anesthesia-induced memory loss, Huntington's Disease, and many others.

In another embodiment, the patient has or is at risk of developing disease-related reduced cognitive function caused by reduced neuronal metabolism, for example, reduced cognitive function associated with Alzheimer's Disease (AD), Parkinson's Disease, Friedreich's Ataxia (FRDA), GLUT1-deficient Epilepsy, Leprechaunism, and Rabson-Mendenhall Syndrome, Coronary Arterial Bypass Graft (CABG) dementia, anesthesia-induced memory loss, Huntington's Disease, and many others.

In another embodiment, the subject, lacks the ApoE4 genotype as described in U.S. Pat. No. 8,445,535, the entirety of which is hereby incorporated by reference.

As used herein, reduced neuronal metabolism refers to all possible mechanisms that could lead to a reduction in neuronal metabolism. Such mechanisms include, but are not limited to mitochondrial dysfunction, free radical attack, generation of reactive oxygen species (ROS), ROS-induced neuronal apoptosis, defective glucose transport or glycolysis, imbalance in membrane ionic potential, dysfunction in calcium flux, and the like.

According to the present invention, high blood ketone levels will provide an energy source for brain cells that have compromised glucose metabolism, leading to improved performance in cognitive function. As used herein, “subject” and “patient” are used interchangeably, and refer to any mammal, including humans that may benefit from treatment of disease and conditions associated with or resulting from reduced neuronal metabolism.

“Effective amount” refers to an amount of a compound, material, or pharmaceutical composition, as described herein that is effective to achieve a particular biological result. Effectiveness for treatment of the aforementioned conditions may be assessed by improved results from at least one neuropsychological test. These neuropsychological tests are known in the art and include Clinical Global Impression of Change (CGIC), Rey Auditory Verbal Learning Test (RAVLT), First-Last Names Association Test (FLN), Telephone Dialing Test (TDT), Memory Assessment Clinics Self-Rating Scale (MAC-S), Symbol Digit Coding (SDC), SDC Delayed Recall Task (DRT), Divided Attention Test (DAT), Visual Sequence Comparison (VSC), DAT Dual Task (DAT Dual), Mini-Mental State Examination (MMSE), and Geriatric Depression Scale (GDS), among others.

The term “cognitive function” refers to the special, normal, or proper physiologic activity of the brain, including, without limitation, at least one of the following: mental stability, memory/recall abilities, problem solving abilities, reasoning abilities, thinking abilities, judging abilities, capacity for learning, perception, intuition, attention, and awareness. “Enhanced cognitive function” or “improved cognitive function” refers to any improvement in the special, normal, or proper physiologic activity of the brain, including, without limitation, at least one of the following: mental stability, memory/recall abilities, problem solving abilities, reasoning abilities, thinking abilities, judging abilities, capacity for learning, perception, intuition, attention, and awareness, as measured by any means suitable in the art. “Reduced cognitive function” or “impaired cognitive function” refers to any decline in the special, normal, or proper physiologic activity of the brain.

In another embodiment, the methods of the present invention further comprise determination of the patients' genotype or particular alleles. In one embodiment, the patient's alleles of the apolipoprotein E gene are determined. It has been found that non-E4 carriers performed better than those with the E4 allele when elevated ketone body levels were induced with MCT. Also, those with the E4 allele had higher fasting ketone body levels and the levels continued to rise at the two hour time interval. Therefore, E4 carriers may require higher ketone levels or agents that increase the ability to use the ketone bodies that are present.

In one embodiment, the pharmaceutical compositions of the disclosure are administered orally. Therapeutically effective amounts of the therapeutic agents can be any amount or dose sufficient to bring about the desired effect and depend, in part, on the severity and stage of the condition, the size and condition of the patient, as well as other factors readily known to those skilled in the art. The dosages can be given as a single dose, or as several doses, for example, divided over the course of several weeks, as discussed elsewhere herein.

The pharmaceutical compositions of the disclosure, in one embodiment, are administered in a dosage required to increase blood ketone bodies to a level required to treat and/or prevent the occurrence of any disease- or age-associated cognitive decline, such as AD, AAMI, and the like. Appropriate dosages may be determined by one of skill in the art.

In one embodiment, oral administration of a pharmaceutical composition of the disclosure results in hyperketonemia. Hyperketonemia, in one embodiment, results in ketone bodies being utilized for energy in the brain even in the presence of glucose. Additionally, hyperketonemia results in a substantial (39%) increase in cerebral blood flow (Hasselbalch, S. G., et al., Changes in cerebral blood flow and carbohydrate metabolism during acute hyperketonemia, Am J Physiol, 1996, 270:E746-51). Hyperketonemia has been reported to reduce cognitive dysfunction associated with systemic hypoglycemia in normal humans (Veneman, T., et al., Effect of hyperketonemia and hyperlacticacidemia on symptoms, cognitive dysfunction, and counterregulatory hormone responses during hypoglycemia in normal humans, Diabetes, 1994, 43:1311-7). Please note that systemic hypoglycemia is distinct from the local defects in glucose metabolism that occur in any disease- or age-associated cognitive decline, such as AD, AAMI, and the like.

Administration can be on an as-needed or as-desired basis, for example, once-monthly, once-weekly, daily, or more than once daily. Similarly, administration can be every other day, week, or month, every third day, week, or month, every fourth day, week, or month, and the like. Administration can be multiple times per day. When utilized as a supplement to ordinary dietetic requirements, the composition may be administered directly to the patient or otherwise contacted with or admixed with daily feed or food.

The pharmaceutical compositions provided herein are, in one embodiment, intended for “long term” consumption, sometimes referred to herein as for ‘extended’ periods. “Long term” administration as used herein generally refers to periods in excess of one month. Periods of longer than two, three, or four months comprise one embodiment of the instant invention. Also included are embodiments comprising more extended periods that include longer than 5, 6, 7, 8, 9, or 10 months. Periods in excess of 11 months or 1 year are also included. Longer terms use extending over 1, 2, 3 or more years are also contemplated herein. “Regular basis” as used herein refers to at least weekly, dosing with or consumption of the compositions. More frequent dosing or consumption, such as twice or thrice weekly are included. Also included are regimens that comprise at least once daily consumption. The skilled artisan will appreciate that the blood level of ketone bodies, or a specific ketone body, achieved may be a valuable measure of dosing frequency. Any frequency, regardless of whether expressly exemplified herein, that allows maintenance of a blood level of the measured compound within acceptable ranges can be considered useful herein. The skilled artisan will appreciate that dosing frequency will be a function of the composition that is being consumed or administered, and some compositions may require more or less frequent administration to maintain a desired blood level of the measured compound (e.g., a ketone body).

Administration can be carried out on a regular basis, for example, as part of a treatment regimen in the patient. A treatment regimen may comprise causing the regular ingestion by the patient of a pharmaceutical composition of the disclosure in an amount effective to enhance cognitive function, memory, and behavior in the patient. Regular ingestion can be once a day, or two, three, four, or more times per day, on a daily or weekly basis. Similarly, regular administration can be every other day or week, every third day or week, every fourth day or week, every fifth day or week, or every sixth day or week, and in such a regimen, administration can be multiple times per day. The goal of regular administration is to provide the patient with optimal dose of a pharmaceutical composition of the disclosure, as exemplified herein.

Dosages of the inventive compositions, such as, for example, those comprising MCT, may be administered in an effective amount to increase the cognitive ability of patients afflicted with diseases of reduced neuronal metabolism, such as in patients with any disease- or age-associated cognitive decline, such as, AD, AAMI, and the like.

Effective amounts of dosages of MCTs, i.e., compounds capable of elevating ketone body concentrations in an amount effective for the treatment of or prevention of a disease, condition or disorder (e.g., the loss of cognitive function caused by reduced neuronal metabolism) will be apparent to those skilled in the art. As discussed herein above, such effective amounts can be determined in light of disclosed blood ketone levels. Where the compound capable of elevating ketone body concentrations is MCT, the MCT dose, in one embodiment, is in the range of about 0.05 g/kg/day to about 10 g/kg/day of MCT. In other embodiments, the dose will be in the range of about 0.25 g/kg/day to about 5 g/kg/day of MCT. In other embodiments, the dose will be in the range of about 0.5 g/kg/day to about 2 g/kg/day of MCT. In other embodiments, the dose will be in the range of about 0.1 g/kg/day to about 2 g/kg/day. In other embodiments, the MCT dose may be at least 5 g/day, at least 10 g/day, at least 15 g/day, at least 20 g/day, at least 25 g/day, at least 30 g/day, at least 35 g/day, at least 40 g/day, at least 45 g/day, at least 50 g/day, at least 55 g/day, at least 60 g/day, at least 65 g/day, at least 70 g/day, at least 75 g/day, at least 80 g/day, etc. In yet other embodiments, the MCT dose may be between 10 g/day and 80 g/day, between 20 g/day and 80 g/day, between 30 g/day and 80 g/day, between 30 g/day and 60 g/day, etc.

In some embodiments, in order to reduce potential safety and tolerability issues that may be associated with high dosages, the final dosage of MCT may be achieved by titrating up to the final therapeutically effective dosage. By way of example, titration may be performed over 1 to 8 weeks, 1 to 6 weeks, 1 to 4 weeks, 2 to 4 weeks, etc., with adjustments in dosage of 1g to 20 g, 2g, to 20g, 5g to 20g, 5 g to 10 g of tricaprilin per week.

Convenient unit dosage containers and/or compositions include sachets or containers of spray dried particles, tablets, capsules, lozenges, troches, hard candies, nutritional bars, nutritional drinks, metered sprays, creams, and suppositories, among others. The compositions may be combined with a pharmaceutically acceptable excipient such as gelatin, oil(s), and/or other pharmaceutically active agent(s). Some examples of compositions are described in WIPO Publication 2008/170235, the entirety of which is incorporated by reference. For example, the compositions may be advantageously combined and/or used in combination with other therapeutic or prophylactic agents, different from the subject compounds. In many instances, administration in conjunction with the subject compositions enhances the efficacy of such agents. For example, the compounds may be advantageously used in conjunction with antioxidants, compounds that enhance the efficiency of glucose utilization, and mixtures thereof.

In some embodiments, the inventive compounds may be administered in the substantial absence of protein, or be co-formulated without protein.

In some embodiments, the MCT formulation may be co-administered with protein, or be co-formulated with protein.

In some embodiments, the MCT formulation may be co-administered with protein, or be co-formulated with protein. Protein can include more than one type of protein or protein different from one or more sources. Appropriate proteins are known in the art. If co-formulated, the amount of protein to use can include at least about 0.1 g, at least about 1g, at least about 10 g, at least about 50 g, at least about 100 g, at least about 150 g, at least about 200 g, at least about 250 g, at least about 300 g, at least about 400 g. Amounts of protein can be at least about 1 g, at least about 50 g, at least about 100 g. The compositions can comprise from about 15% to about 40% protein, on a dry weight basis. Sources of such proteins include legumes, grains, dairy, nuts, seeds, fruits, vegetables, animals, insects, synthetic sources (e.g., genetically modified yeast), or mixtures thereof. The compositions also optionally comprise other components that comprise protein such as dried whey and other dairy products or by-products. In some embodiments the MCT formulations are administered in the presence of protein-based drinks (e.g., Ensure and similar protein-based drink and nutrition supplements).

Additionally, in some embodiments, the MCT formulation may be co-administered with carbohydrate, or be co-formulated with carbohydrate. Carbohydrate can include more than one type of carbohydrate. Appropriate carbohydrates are known in the art, and include simple sugars, such as glucose, fructose, sucrose, and the like, from conventional sources such as corn syrup, sugar beet, and the like. If co-formulated, the amount of carbohydrate to use can include at least about 0.1 g, at least about 1g, at least about 10 g, at least about 50 g, at least about 100 g, at least about 150 g, at least about 200 g, at least about 250 g, at least about 300 g, at least about 400 g. Amounts of carnitine can be at least about 1 g, at least about 50 g, at least about 100 g. The compositions can comprise from about 15% to about 40% carbohydrate, on a dry weight basis. Sources of such carbohydrates include grains or cereals such as rice, corn, sorghum, alfalfa, barley, soybeans, canola, oats, wheat, or mixtures thereof. The compositions also optionally comprise other components that comprise carbohydrates such as dried whey and other dairy products or by-products.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Rat Model for Formulation Development

Background: In order to rapidly screen formulations of tricaprilin, the rat as pharmacokinetic (PK) model of human formulations has been investigated. PK studies are performed to evaluate the absorption, distribution, metabolism and excretion (ADME) in animals. PK results allow us to define dose, dosing frequency, route of dosing and onset of action.

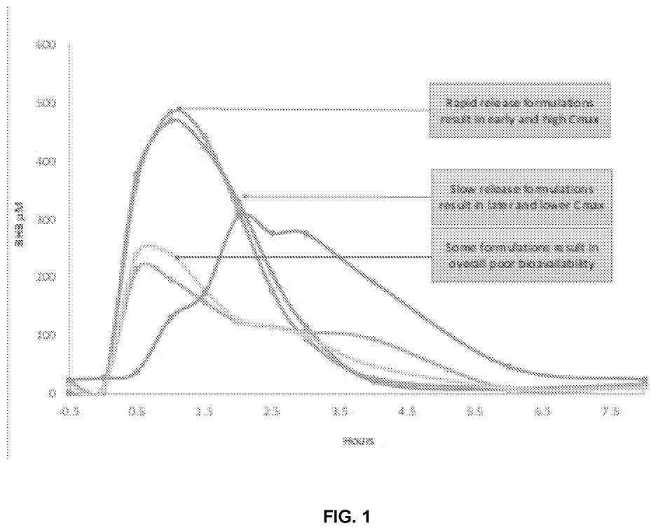

Methods: Several formulations of tricaprilin have been studied in human PK studies and found several differences in Cₘₐₓ, Tₘₐₓ and AUC related to the release from the formulation (as described herein and shown in FIG. 1).

In the present study, the PK profiles of these same formulations were investigated in rat to determine if rats qualitatively replicated the human results. Healthy young adult male Sprague Dawley rats were used as a test system for this PK study. Five animals per group were used, animals were between 9 to 12 weeks old and the weight variation of animals did not exceed ±20% of the mean weight.

Animals were dosed by oral gavage at Biological Resource Centre (BRC), Agency for Science, Technology and Research (A*STAR), Singapore. Sample analysis was performed by Agilex Biolabs Pty Ltd (Thebarton SA, Australia). Concentrations of acetoacetate and β-hydroxybutyrate in rat serum were determined by LC/MS. Ketone body concentrations (pM) were calculated as a sum of acetoacetate (μM) and β-hydroxybutyrate (μM) concentrations. Ketone body data was analyzed using WinNonlin.

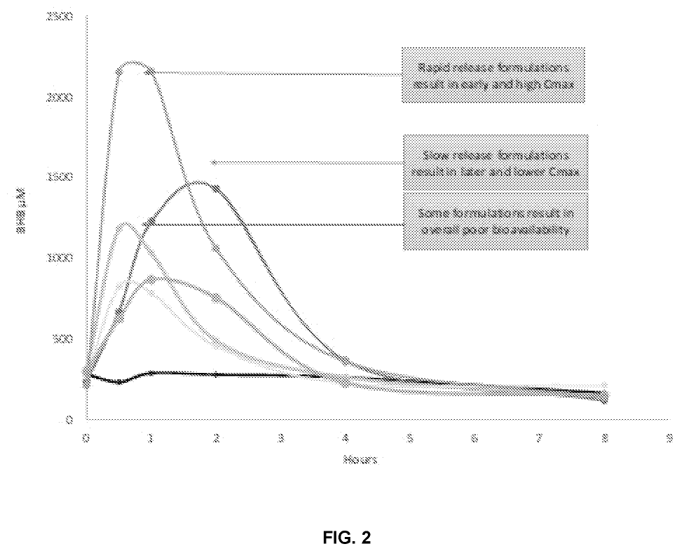

Results: Rats qualitatively mirrored results from human studies. Formulations that exhibited slow release in humans similarly were found to be slow releasing in SD rats. Formulations that exhibited fast release in humans were found to be fast releasing in SD rats. Results of rat studies are shown in FIG. 2 and Table 1 below.

TABLE 1

| | Tmax | | Cmax | | AUCall | |
|---|---|---|---|---|---|---|
| Formula | Mean | SD | Mean | SD | Mean | SD |
| A | 0.70 | 0.27 | 2361.00 | 539.26 | 5698.01 | 1140.76 |
| B | 1.10 | 0.55 | 935.60 | 261.29 | 3158.83 | 564.57 |
| C | 0.90 | 0.65 | 902.60 | 557.51 | 2860.42 | 432.80 |
| D | 0.70 | 0.27 | 1302.20 | 413.49 | 3255.25 | 762.79 |
| E | 1.60 | 0.55 | 1717.00 | 780.38 | 4800.10 | 1250.55 |
| Control | 1.40 | 1.67 | 445.91 | 224.53 | 1945.54 | 1002.00 |

Conclusion: In preliminary studies, rats represent a model for tricaprilin formulation development.

### Example 2—PK-PD Modeling and Simulation

Background: This example set out to determine what doses would be expected to produce maximal results, based on optimal filling of the ‘metabolic gap’ which has been identified in Alzheimer's disease (AD) subject brains on dual tracer (FDG-Acetoacetate) PET imaging. This metabolic gap represents the gap between energy consumption in healthy young brain cells vs AD brain cells. “Filling the gap” is correlated with improved cognition.

Methods: Using advanced analytics and pharmacology modeling, a PK-PD model was developed to fit available data which included cerebral metabolic rate data from ingestion of MCTs. Following development of the model, simulations were run to determine doses required to fill 25-50% of the metabolic gap.

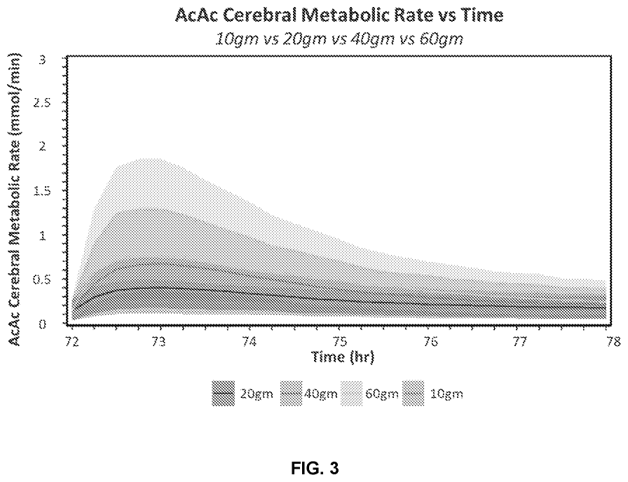

Results: To ‘Fill the Gap’, more than 20 g caprylic triglyceride is required. Our goal is to fill 25-50% of the metabolic gap to ensure a clinical effect. With reference to FIG. 3, 60 g per day of tricaprilin is the targeted dose.

### Example 3—PK Studies of Optimized Formulations of Tricaprilin

Part 1:

A Phase 1, Randomised, Single-Center, Single-Dose, Placebo-Controlled, 3-Way Crossover Study to Compare the Pharmacokinetics, Safety and Tolerability of a Lipid Multi-Particulate (LMP) Formulation and Spray-Dried (SD) Formulations of Tricaprilin (TC) on Ketone body Production.

Objective:

Primary Objectives:

To assess the safety and tolerability of single-dose administration of each of 2 tricaprilin formulations (AC-SD-03 and AC-LMP-01) and the placebo formulation, AC-SD-03P, in healthy, male volunteers.

To compare ketone body levels (i.e., total ketones, β-hydroxybutyrate [BHB], acetoacetate [AcAc]), tricaprilin and octanoic acid levels after single-dose administrations of each of the tricaprilin formulations, AC-SD-03 and AC-LMP-01, and the placebo formulation, AC-SD-03P in healthy, young, male volunteers.

Secondary/Exploratory Objective

To assess the effects of APOE4 status on tricaprilin BA, metabolism and ketone body production,

Methodology:

This was an open label, randomised, 3 way crossover, pilot pharmacokinetic (PK), safety and tolerability study, to assess safety and tolerability and to compare the ketone body levels (i.e., total ketones, BHB, AcAc), tricaprilin and octanoic acid levels after single-dose administrations of each of the tricaprilin formulations and, AC-SD-03 and AC-LMP-01, and the placebo formulation, AC SD-03P and to assess the effects of APOE4 status on tricaprilin BA, metabolism and ketone body production in healthy, male volunteers under fed conditions.

Twelve (12), healthy, adult male subjects were enrolled to be dosed in of the 2 cohorts.
Cohort 1: comprising Chinese subjects (n=6)
Cohort 2: comprising subjects from non-Chinese (Caucasian) ethnic population (n=6)

Both cohorts were conducted concurrently. On Day 1 of Period 1, subjects were randomised to 1 of 6 treatment sequences.

On Day 1 of Periods 1, 2, and 3, 30′ minutes following completion of breakfast, subjects received a single oral dose of AC SD-03, AC-LMP-01, and AC-SD-03P. Subjects received each treatment on one occasion. Blood samples for PK sampling to measure total ketones, BHB, AcAc, octanoic acid and tricaprilin would be taken pre-dose and up to 24-hours following dosing. Prior to entering the trial, subjects had a screening visit to establish eligibility within 28 days before Day −1 of Period 1. Upon arrival for confinement, subjects were randomized to receive a single-dose of study medication (AC-SD-03 and AC-LMP-01) or placebo (AC-SD-03P) (1:1:1 active to placebo) in accordance with the randomization scheme generated by Syneos. There was a washout period of 2 days between doses. Subjects were housed on Day −1 of Period 1, at the time indicated by the Clinical research facility (CRU) until after the 24 hour blood draw on Day 1 of Period 3. The total study duration (not including Screening but including the 3-day Follow-up period) was 11 days.

Diagnosis and Main Criteria for Inclusion:

Subjects had to be healthy, male/adult non-smokers, aged 18 and 50 years (inclusive), with body mass index (BMI) ≥18.0 and <32.0 kg/m2. All subjects had to be in compliance with the inclusion and exclusion criteria described in the protocol and were judged eligible for enrolment in this study based on medical and medication histories, demographic data (including sex, age, race, ethnicity, body weight [kg], height [cm], and BMI [kg/m2]), vital signs measurements, a 12 lead electrocardiogram (ECG), a physical examination, a urine drug screen, an alcohol breath test, and clinical laboratory tests (serum chemistry, hematology, urinalysis, human immunodeficiency virus [HIV], hepatitis C [HCV] antibodies, and hepatitis B surface antigen [HBSAg] Hepatitis B core antigen [HCsAg], Thyroid stimulating hormone (TSH), and Hemoglobin A1c tests).

Treatment Protocol: The following formulations were administered, with the following treatment regimens:

| | Study Drugs | | |
|---|---|---|---|
| | | | Treatment C |
| Parameters | Treatment A | Treatment B | (Placebo) |
| Product | AC-SD-03 | AC-LMP-01 | AC-SD-03P |
| Strength | 50 g AC-SD-03 | 50 g AC-LMP-01 | 50 g AC-SD-03 |
| | (equivalent to | (equivalent to | (containing 20 |
| | 20 g tricaprilin) | 20 g tricaprilin) | g of safflower oil) |
| Dosage form | Spray dried | Lipid Multi- | Spray dried |
| | powder | particulate | powder |
| | | powder | |
| Dose | 1 × 50 g | 1 × 50 g | 1 × 50 g |
| administered | | | |
| Route of | Oral | Oral | Oral |
| administration | | | |

| Treatment | Description |
|---|---|
| Treatment A: | AC-SD-03 (dose to deliver 20 g tricaprilin, approximately |
| | 50 g) at Hour 0 on Day 1 administered approximately 30 |
| | minutes after the start of a standard breakfast. |
| Treatment B: | AC-LMP-01 (dose to deliver 20 g tricaprilin, approximately |
| | 50 g) at Hour 0 on Day 1 administered approximately 30 |
| | minutes after the start of a standard breakfast. |
| Treatment C: | AC-SD-03P (matching placebo to AC-SD-03, |
| | approximately 50 g) at Hour 0 on Day 1 administered |
| | approximately 30 minutes after the start of a standard |
| | breakfast. |

Blood Sampling Points: In each period, a total of 13 blood samples were drawn from each subject for PK analyses of ketone body levels (i.e., total ketones, BHB, AcAc), tricaprilin and octanoic acid. Blood samples were collected at −1 hours, 0 hour (predose) and at 0.5. 1.0, 1.5, 2.0, 2.5, 3.0, 4.0, 6.0, 8.0, 12 and 24 hours after dosing.

Criteria for Evaluation:

Safety:

Treatment-emergent adverse events (TEAEs), serious adverse events (SAEs), laboratory parameters (serum chemistry, hematology, and urinalysis), 12-lead ECG, physical examination including weight, gastrointestinal side effect, and vital signs assessments.

Pharmacokinetic:

The following PK parameters were to be calculated for ketone body levels (i.e., total ketones, BHB, AcAc), tricaprilin level and octanoic acid level (unadjusted and baseline-adjusted), using standard non-compartmental methods: AUC₀₋ₜ, AUC₀₋₄, AUC₀₋₆, AUC₀₋₈, AUC₀₋₂₄, AUC_{0-inf}, AUC_{%extrap}, Tₘₐₓ, Kₑₗ, t_{1/2} and Cₘₐₓ.

Parametric ANOVA (Linear Mixed Model) and geometric confidence intervals for treatment comparisons A/B, A/C and B/C on AUC₀₋ₜ, AUC₀₋₄, AUC₀₋₆, AUC₀₋₈, AUC₀₋₂₄, AUC_{0-inf} (if calculated), and Cₘₐₓ for unadjusted and baseline adjusted data;

Factors in the ANOVA model: Sequence, Subject within Sequence, Period and Treatment;

Ln-transformed parameters: AUC₀₋ₜ, AUC₀₋₄, AUC₀₋₆, AUC₀₋₈, AUC₀₋₂₄, AUC_{0-inf} (if calculated), and Cₘₐₓ.

Statistical Methods:

Safety Analyses:

Demographic parameters were summarized descriptively. Demographic and baseline characteristics (including gender, age, race, ethnicity, smoking history, height, weight and BMI) were summarised by randomised treatment sequence and overall.

The Medical Dictionary for Regulatory Activities® (MedDRA®) Version 22.0 was used to classify all AEs reported during the study by System Organ Class (SOC) and Preferred Term (PT).

TEAEs were summarised by actual treatment. The number and percentage of subjects experiencing AEs and the number of TEAEs were tabulated. Subjects who experienced the same AE (in terms of MedDRA preferred term) more than once was only counted once for that event, however, the total number of events were also be counted per category. This also applies to sub-categories displayed in the summaries.

The relationship for each TEAE was classified according to the study protocol as likely, probably, possibly, unlikely, or unrelated to study drug. The severity of TEAEs were classified according to the study protocol as mild, moderate, or severe.

The following summaries were presented:
Overall summary of TEAEs
TEAEs by SOC and PT
TEAEs by SOC, PT, and severity
TEAEs by SOC, PT, and relationship to study drug
Serious TEAEs by SOC and PT

Laboratory data (hematology and serum chemistry) was summarised at each protocol scheduled visit, by actual treatment. Actual values and actual changes from baseline was presented.

In addition, a shift table representing the categorical change of laboratory range results (low, normal, high) from baseline to each post baseline visit was presented.

Urinalysis results evaluation was summarised at each protocol-scheduled time point, by actual treatment, using frequency tabulations.

Vital sign measurements were summarised at each protocol-scheduled time point, by actual treatment. Actual values and actual changes from baseline was presented.

ECG values were summarised at each protocol-scheduled visit, by actual treatment. Actual values and actual changes from baseline was presented. In addition, a shift table representing the categorical change of ECG results (normal, abnormal not clinically significant, or abnormal clinically significant) from baseline to each post baseline visit was presented.

For the Pain Numerical Rating Scale and Baxter Retching Faces Scale results, average scores were summarized at each protocol-scheduled visit/time point, by actual treatment. Actual values and actual changes from baseline were presented.

Pharmacokinetic Analyses:

Individual concentration versus time curves were presented using linear scale for each analyte sorted by treatment. Mean concentration versus time curves were presented for both linear and semi-log scales for each analyte sorted by treatment.

Unadjusted and baseline-adjusted PK concentrations of total ketones, BHB, AcAc, tricaprilin and octanoic acid were listed and summarized, by nominal sampling time, and cohort/actual treatment. Descriptive statistics (arithmetic and geometric means, standard deviation [SD], arithmetic and geometric coefficients of variation [CV %], minimum [Min], maximum [Max], and median) of the ketone body levels (i.e., total ketones, BHB, AcAc), tricaprilin and octanoic acid concentrations versus time was presented as well for the PK parameters sorted by treatment.

Results

Pharmacokinetic:

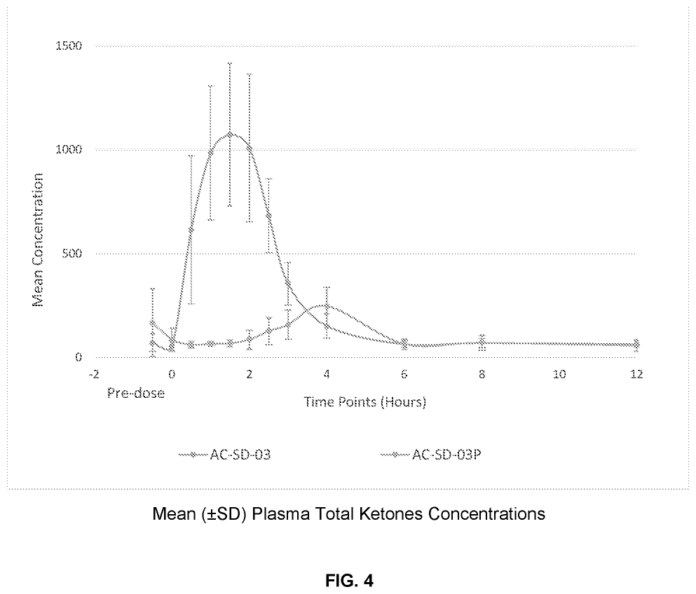

With reference to FIG. 4, mean (±SD) plasma total ketones concentrations are shown. Generally, ketone body levels (AUC total ketones, BHB, AcAc) were comparable (or higher in some cases) for Treatment A (AC-SD-03) compared to Treatment B (AC-LMP-01). Ketone body levels were significantly higher for Treatment A (AC-SD-03) and Treatment B (AC-LMP-01) compared to the placebo formulation, Treatment C (AC-SD-03P).

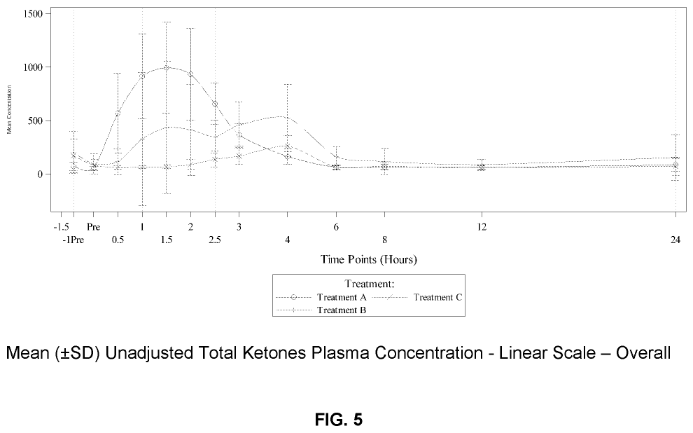

With reference to FIG. 5, mean (±SD) unadjusted total ketones plasma concentrations, linear scale, overall are shown. As shown, −1Pre-dose: “−1 HOUR PRE-BREAKFAST”; Pre-dose: “0 HOUR PRE-DOSE”; Treatment A: AC-SD-03, dose to deliver 20 g tricaprilin, approximately 50 g dose equivalent to 20 g tricaprilin; Treatment B: AC-LMP-01, dose to deliver 20 g tricaprilin, approximately 50 g dose equivalent to 20 g tricaprilin; and Treatment C: AC-SD-03P, matching placebo to AC-SD-03, approximately 50 g.

Mean unadjusted total ketones Cₘₐₓ overall for AC-SD-03 (Treatment A) and AC-LM P-01 (Treatment B) were respectively 1043.6 μM (CV % 39.2) and 632.0 μM (CV % 70.5) while was 258.7 μM (CV % 38.0) for Treatment C (AC-SD-03P). Based on these results, it can be concluded that Treatments A and B presented concentrations greater than 500 μM and therefore confirmed the ketogenic status of AC-SD-03 and AC-LMP-01. Overall, median Tₘₐₓ occurred around 1.5 h, 3.4 h and 4.0 h post-dose for Treatments A, B and C, respectively.

Following the administration of AC-SD-03 20 g (Treatment A), high variability in PK parameters was observed in Cohort 1 (Chinese population) for total ketones, BHB and tricaprillin (CV % 50-60%) compared to Cohort 2 (Caucasian population) (CV % 18-20%). According to the alternative analysis excluding Subject 037 who presented outlier results for ketone body levels, the variability decreased for Cohort 1, which had the effect of reducing the difference between the two populations for total ketone levels (CV % 40%). However, the principal root cause of these results obtained for Subject 037 was not clearly identified.

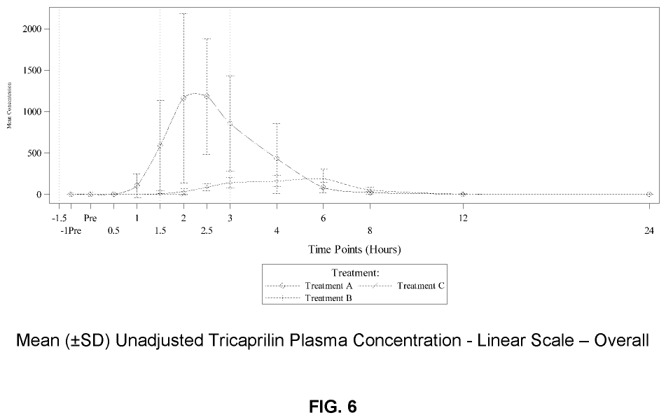

With reference to FIG. 6, mean (±SD) unadjusted tricaprilin plasma concentrations, linear scale, overall, are shown. As shown, −1Pre-dose: “−1 HOUR PRE-BREAKFAST”; Pre-dose: “0 HOUR PRE-DOSE”; Treatment A: AC-SD-03, dose to deliver 20 g tricaprilin, approximately 50 g dose equivalent to 20 g tricaprilin; Treatment B: AC-LMP-01, dose to deliver 20 g tricaprilin, approximately 50 g dose equivalent to 20 g tricaprilin; and Treatment C: AC-SD-03P, matching placebo to AC-SD-03, approximately 50 g.

The rate and extent of absorption of tricaprilin are significantly greater (3- to 6-fold, respectively) following an oral single administration of AC-SD-03 20 g (Treatment A) compared to AC-LMP-01 20 g (Treatment B). The mean T_{1/2 el} was 2.4 hours for Treatment A and 2.1 hours for Treatment B. The median Tₘₐₓ occurred around 2.5 hours for Treatment A and around 4 hours post-dose for Treatment B (overall population).

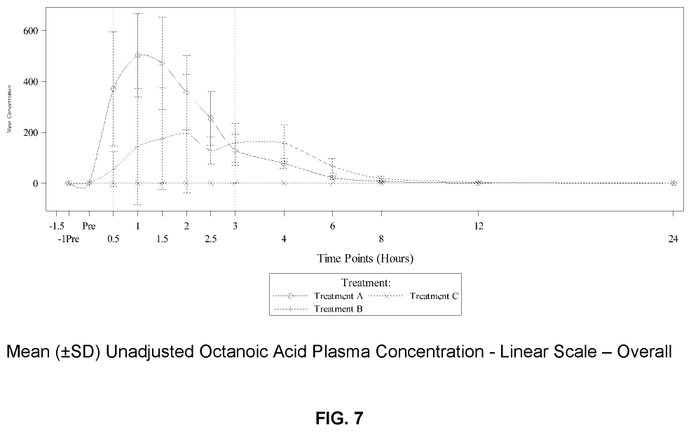

With reference to FIG. 7, mean (±SD) unadjusted octanoic acid plasma concentrations, linear scale, overall, are shown. As shown, −1Pre-dose: “−1 HOUR PRE-BREAKFAST”; Pre-dose: “0 HOUR PRE-DOSE”; Treatment A: AC-SD-03, dose to deliver 20 g tricaprilin, approximately 50 g dose equivalent to 20 g tricaprilin; Treatment B: AC-LMP-01, dose to deliver 20 g tricaprilin, approximately 50 g dose equivalent to 20 g tricaprilin; and Treatment C: AC-SD-03P, matching placebo to AC-SD-03, approximately 50 g.

For octanoic acid levels, no comparison was possible between the two test treatments, AC-SD-03 20 g (Treatment A) and AC-LMP-01 20 g (Treatment B), and the placebo formulation AC-SD-03P (Treatment C) since the concentrations were all below the limit of quantitation for 11 of 12 subjects enrolled in the study.

The effects of Apolipoprotein E 4 (APOE4) status on tricaprilin bioavailability, metabolism and ketone body production cannot be determined in the current study since all subjects were APOE4 negative.

Safety and Tolerability:

With reference to the table below, a total of 9 TEAEs were reported by 8 (66.7%) of the 12 subjects who received at least one dose of the study medication (safety population). The frequency of subjects who reported TEAEs overall was at least 7-fold higher in subjects who received Treatment A (58.3%) when compared to Treatment B (8.3%). No TEAEs were reported by subjects after receiving Treatment C (Placebo). The frequency of subjects who reported TEAEs was similar between Chinese and Caucasian subjects for all treatments. The most frequently reported TEAE was Nausea, reported in 5 subjects after receiving Treatment A (3 Caucasian and 2 Chinese subjects). All TEAEs reported were mild in severity and were considered as related to the study drug. There were no deaths during the study and none of the TEAEs reported was severe or serious. No TEAEs lead to subject discontinuation after dosing.

| | SOC | | |
|---|---|---|---|
| | Treatment A | Treatment B | Treatment C |
| Preferred Term | AC-SD-03 | AC-LMP-01 | AC-SD-03P |
| Number of subjects dosed, N | 12 | 12 | 12 |
| Number of TEAEs, n | 8 | 1 | 0 |
| Number of subjects with | 7 (58.3%) | 1 (8.3%) | 0 |
| TEAEs, N (%) | | | |
| Gastrointestinal disorders | 7 (58.3%) | 1 (8.3%) | 0 |
| Nausea | 5 (41.7%) | 0 | 0 |

There were no TEAEs related to clinical laboratory results, vital signs, and ECG results. No relevant differences were observed between the treatment groups and between Chinese and Caucasian subjects with respect to mean values and changes from baseline for clinical laboratory results, vital signs, and ECG results.

Most subjects had a Pain Numerical Rating Scale (NRS) result of 0 on rating scale during the study. Few subjects had a Pain NRS of 1 to 3 within 3 hours after administration of Treatment A or B. Most subjects had a Baxter Retching Faces (BARF) Scale result ≤4 on rating scale during the study. BARF Scale results of 1 or higher were reported mostly within 2 hours after administration of Treatment A. There were no relevant differences between Chinese and Caucasian subjects for Pain NRS and BARF scale results.

Conclusions:

Safety:

Overall, both tricaprilin formulations (AC-SD-03 and AC-LMP-01) and the placebo formulation were well tolerated, apart from expected mild GI symptoms, in healthy, male volunteers, with no major safety concerns, when administered as a single dose equivalent to 20 g tricaprilin or safflower oil, without titration. No GI adverse events were reported with the placebo formulation (AC-SD-03P, Treatment C) containing the same excipients as Treatment A (AC-SD-03) with the replacement of tricaprilin with safflower oil.

Pharmacokinetic:

A single-dose of the AC-SD-03 formulation (containing 20 g tricaprilin) in 12 healthy volunteers resulted in low tricaprilin concentrations (1 μM) peaking at 2.5 hours, whereas the breakdown product and primary absorbed compound, octanoic acid, peaked at approximately 500 μM after 1 hour. This led to a ketone body response with Tₘₐₓ 1.5 h, and Cₘₐₓ of 1 mM (levels of BHB to AcAc were approximately 3.5:1). The T_{1/2 el} was 2.4 h, and ketone body levels returned to baseline levels after 4 hours.

There was no statistically significant difference between Caucasian and Chinese subjects. The AC-LMP-01 formulation (containing 20 g tricaprilin) had a slower release profile, with overall similar total ketones AUC_{0-inf} to AC-SD-03 but a lower Cₘₐₓ (632 μM) and longer Tₘₐₓ (3.4 h). The alternative analysis (excluding Subject 037) reinforced the above conclusions. The placebo formulation AC-SD-03P was not ketogenic.

Part 2:

A Phase 1, Randomised, Single-Center, Single-Dose, Placebo-Controlled, 3-Way Crossover Study to Compare the Pharmacokinetics, Safety and Tolerability of a Lipid Multi-Particulate (LMP) Formulation and Spray-Dried (SD) Formulations of Tricaprilin (TC) on Ketone body Production. Part 2 to Include a 2-way Crossover to Compare the Pharmacokinetics, Safety and Tolerability of Two Spray-dried (SD) Formulations of Tricaprilin (TC) on Ketone Body Production (Part 2).

Objective:

To assess the safety and tolerability of single-dose administration of each of tricaprilin formulations (AC-SD-03, Manufactured at Anthem Bioscience Pvt. Ltd., India and AC-1202), in healthy, male volunteers.

To compare ketone body levels (i.e., total ketones, β-hydroxybutyrate [BHB], acetoacetate [AcAc]), tricaprilin and octanoic acid levels after single-dose administrations of each of the tricaprilin formulations, AC-SD-03 (Anthem) and AC-1202, in healthy, young, male volunteers.

Methodology:

After completion of Part 1, an addendum to the protocol was prepared to include a 2-way crossover study to compare the pharmacokinetic (PK), safety, and tolerability of two spray-dried (SD) formulations of tricaprilin on ketone body production (referred as Part 2).

These are the main changes between Part 1 and Part 2 of this study:

Subjects received two formulations of study products in Part 2;

The AC-SD-03 formulation used in Part 2 was manufactured at a different manufacturing site than the site of manufacture for the AC-SD-03 formulation used in Part 1;

12-Lead electrocardiogram (ECG) and safety laboratory analysis were only performed at the screening visit in Part 2;

The apolipoprotein E gene 4 (APOE4) status of subjects was not determined in Part 2;

The measurement of gastrointestinal side effects with the pain numeric rating scale (NRS) and Baxter Retching faces (BARF) scales was not performed in Part 2.

Twenty (20), healthy, adult male subjects (Chinese and non-Chinese) were to be enrolled in Part 2. These could be the same subjects who participated in Part 1 or new subjects.

Cohort 1: Chinese subjects (a minimum of 10 was specified)

Cohort 2: from non-Chinese ethnic population

Additional Chinese subjects could be enrolled in Cohort 1, in place of subjects in Cohort 2, in order to maximise the number of Chinese subjects into the study.

On Day 1 of Period 1, subjects were randomised to one of two treatment sequences. On Day 1 of Periods 1 and 2, following completion of a protocol specified standard breakfast, subjects received a single oral dose of AC-SD-03 or AC-1202. Subjects received each treatment on one occasion. Blood samples for PK sampling to measure total ketones, BHB, acetoacetate, octanoic acid and tricaprilin were taken pre-dose and up to 8 hours following dosing.

There was a washout period of 2 days between doses. The total study duration (not including Screening but including the 3-day Follow-up period) was 8 days.

Except for the changes described above, there were no changes in the study conduct in regards to study procedures, safety monitoring, confinement, and follow-up compared to Part 1.

Diagnosis and Main Criteria for Inclusion:

Subjects had to be healthy, male/adult non-smokers, aged 18 and 50 years (inclusive), with body mass index (BMI) ≥18.0 and <32.0 kg/m². There were no changes in selection criteria between Part 1 and Part 2.

Treatment Protocol: The following formulations were administered, with the following treatment regimens:

Treatment

| | Study Drugs | |
|---|---|---|
| Parameters | Treatment D | Treatment E |
| Product | AC-SD-03 | AC-1202 |
| Strength | 50 g AC-SD-03 (equivalent | 60 g (equivalent |
| | to 20 g tricaprilin) | to 20 g tricaprilin) |
| Dosage form | Spray-dried powder | Spray-dried powder |
| Dose administered | 1 × 50 g | 1 × 60 g |
| Route of | Oral | Oral |
| administration | | |
| Manufacturer | Anthem Bioscience Pvt. Ltd. | SensoryEffects |
| Lot/Batch No. | A222000035 | WFV2618 |

| Treatment | Description |
|---|---|
| Treatment D: | AC-SD-03 (dose to deliver 20 g tricaprilin, approximately |
| | 50 g) at Hour 0 on Day 1 administered approximately 30 |
| | minutes after the completion of the protocol standard |
| | breakfast. |
| Treatment E: | AC-1202 (dose to deliver 20 g tricaprilin), approximately |
| | 60 g) at Hour 0 on Day 1 administered approximately 30 |
| | minutes after the completion of the protocol standard |
| | breakfast. |

Criteria for Evaluation:

Safety and Tolerability:

Safety was monitored through vital sign measurements, clinical laboratory tests, adverse events (AEs), and physical examination.

Pharmacokinetic:

The following main PK parameters were calculated for total ketones, BHB, acetoacetate, octanoic acid and tricaprilin: AUC0-t, AUC0-4, Cₘₐₓ, and Tₘₐₓ. If appropriate, AUC0-inf, AUC % Extrap, Kel, and T½ were computed.

PK parameters were derived from concentrations by non-compartmental analysis using actual times.

Descriptive statistics (arithmetic and geometric means, standard deviation [SD], coefficient of variation [CV %], minimum [Min], maximum [Max], and median) were presented for the total ketones, BHB, acetoacetate, octanoic acid and tricaprilin concentrations versus time and PK parameters.

Using Generalized Linear Model (GLM) procedures in Statistical Analysis System (SAS), an analysis of variance (ANOVA) was performed, for unadjusted and baseline adjusted, as appropriate, on the natural log (In)-transformed AUC0-t, AUC0-4, AUC0-inf (if computed), and Cₘₐₓ, at the alpha level of 0.05.

The ratio of geometric means (A/B) and 90% confidence interval for the ratio of geometric means, based on least squares means from the ANOVA of the In-transformed data, were calculated for AUC0-t, AUC0-4, AUC0-inf (if computed), and Cₘₐₓ.

Statistical Methods:

There were no changes in the analysis plan between Part 1 and Part 2.

Results

Safety and Tolerability:

With reference to the table below, a total of 28 TEAEs were reported by 17 (81.0%) of the 21 subjects who received at least one dose of the study medication. Eleven subjects (52.4%) reported a TEAE after receiving AC-SD-03 (Treatment D) and 12 subject (60.0%) reported a TEAE after receiving AC-1202 (Treatment E). The frequency of subjects who reported TEAEs was lower in Caucasian than in Chinese for both treatments.

| | SOC | | | |
|---|---|---|---|---|
| | AC-SD-03 | | AC-1202 | |
| Preferred Term | Treatment D | | Treatment E | |
| Number of subjects dosed, N | 21 | | 20 | |
| Number of TEAEs, n | 13 | | 15 | |
| Number of subjects with TEAEs, n (%) | 11 | (52.4) | 12 | (60.0) |
| Gastrointestinal disorders | 11 | (52.4) | 12 | (60.0) |
| Abdominal distension | 5 | (23.8) | 8 | (40.0) |
| Abdominal discomfort | 3 | (14.3) | 4 | (20.0) |
| Nausea | 3 | (14.3) | 1 | (5.0) |

The most commonly reported TEAEs during this study were all related to the SOC gastrointestinal disorders. The most frequently reported TEAEs reported were abdominal distension, abdominal discomfort, and nausea. Gastrointestinal AEs are expected with the use of tricaprilin.

The most frequently reported TEAE was abdominal distension, reported in 5 subjects (23.8%) after receiving AC-SD-03 (2 Chinese and 3 Caucasian subjects) and 8 subjects (40.0%) after receiving AC-1202 (5 Chinese and 3 Caucasian subjects). All TEAEs reported were mild in severity and were considered related to the study drug. There were no deaths and none of the TEAEs reported was severe or serious. No TEAEs led to subject discontinuation after dosing.

There were no TEAEs related to vital signs and no relevant differences were observed between treatments and between Chinese and Caucasian subjects.

Pharmacokinetics:

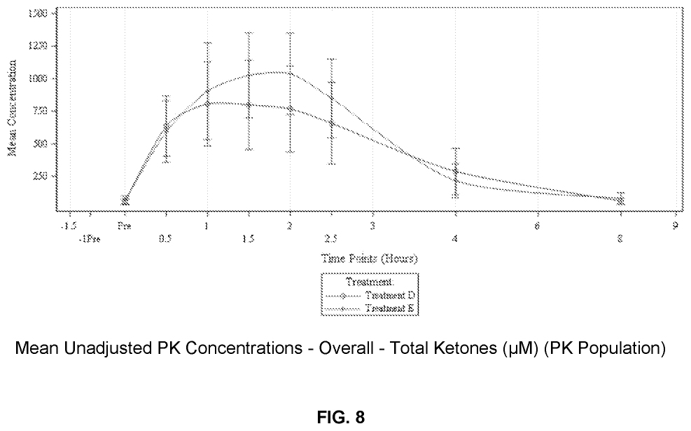

With reference to FIG. 8, mean unadjusted PK concentrations, overall, total ketones (μM) (PK population) are shown. As shown, −1Pre: “−1 hour pre-breakfast”; Pre: “0 hour pre-dose”; Treatment D: AC-SD-03; Treatment E: AC-1202. Based on AUC and Cₘₐₓ, the ketone body levels (total ketones, BHB, AcAc) were generally higher after administration of the AC-1202 formulation than the AC-SD-03 formulation. Indeed, the maximum concentration reached was statistically higher for the AC-1202 than the AC-SD-03 formulations for total ketones [AC-1202: 1111.56 μM (CV % 28.79) vs. AC-SD-03: 917.32 μM (CV % 32.44), p=0.001] (FIG. 8), for BHB [AC-1202: 822.34 μM (CV % 28.72) vs. AC-SD-03: 675.69 μM (CV% 32.73), p=0.001), and for AcAc [AC-1202: 292.26 μM (CV % 33.24) vs AC-SD-03: 241.41 μM (CV % 33.74), p=0.008). Even though statistically different, the administration of AC-SD-03 or AC-1202 resulted in ketone body concentrations greater than 500 μM and therefore confirmed the ketogenic status of both formulations.

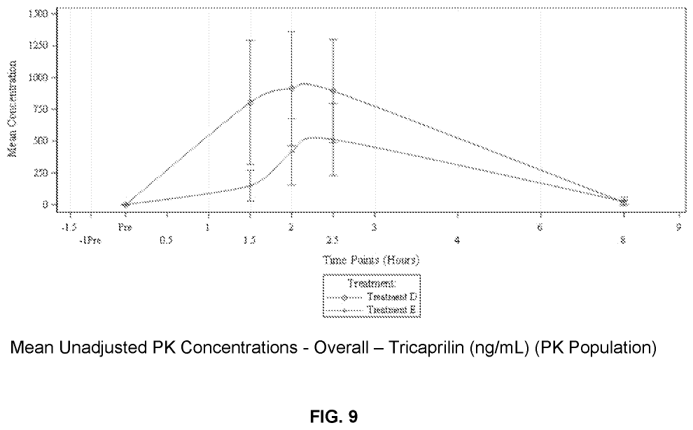

Conversely, the tricaprilin body level was statistically significantly lower after administration of AC-1202 formulation than after AC-SD-03 formulation as measured with the Cₘₐₓ [AC-1202: 478.90 ng/mL (CV % 57.14) vs. AC-SD-03: 940.80 ng/mL (CV % 54.16), p<0.0001] (FIG. 9). With reference to FIG. 9, mean unadjusted PK concentrations, overall, total tricaprilin (ng/mL) (PK population) are shown. As shown, −1Pre: “−1 hour pre-breakfast”; Pre: “0 hour pre-dose”; Treatment D: AC-SD-03; Treatment E: AC-1202.

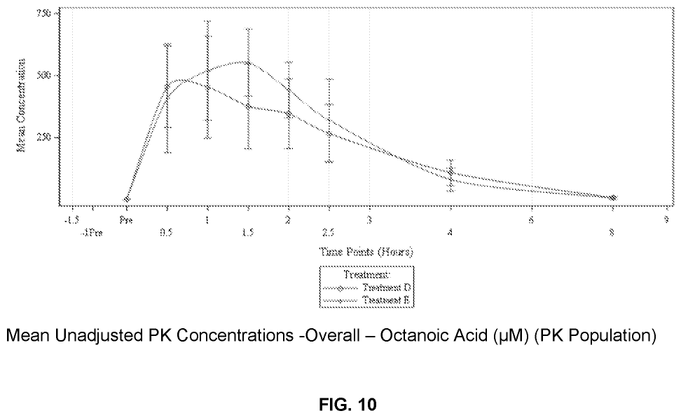

The level of the primary absorbed compound octanoic acid was also higher after administration of the AC-1202 formulation than the AC-SD-03 formulation (FIG. 10). With reference to FIG. 10, mean unadjusted PK concentrations, overall, total octanoic acid (μM) (PK population) are shown. As shown, −1Pre: “−1 hour pre-breakfast”; Pre: “0 hour pre-dose”; Treatment D: AC-SD-03; Treatment E: AC-1202.The maximum concentration reached was statistically higher for the AC-1202 formulation than the AC-SD-03 formulations [AC-1202: 604.18 μM (CV % 31.47) vs. AC-SD-03: 528.91 μM (CV % 31.38), p=0.046].

There was no difference between the two formulations in the median time at which the maximum concentration (Tₘₐₓ) of total ketones and BHB (1.5 h for both) was reached. However, the median Tₘₐₓ was slightly longer to reach with AC-1202 than with AC-SD-03 for AcAc (AC-1202: 1.734 h vs. AC-SD-03: 1.5 h), tricaprilin (AC-1202: 2.5 h vs. AC-SD-03: 2.25 h), and octanoic acid (AC-1202: 1.5 h vs. AC-SD-03: 1.0 h).

The total ketone level measured following administration of AC-SD-03 in terms of AUC and Cₘₐₓ was approximately 0.9 and 0.8 times the level measured after administration of AC-1202 formulation, respectively. Based on the point estimates D/E between 82% and 92%, ketone body levels (AUC total ketones, BHB, AcAc) were comparable for AC-SD-03 and AC-1202. On the other hand, the tricaprilin level in terms of AUC and Cₘₐₓ was approximately 1.7 and 2.0 times higher following AC-SD-03 administration than AC-1202 administration, respectively. Ratios measured for BHB, AcAc, and octanoic acid were similar to those measured for total ketones.

Variations in Cₘₐₓ and AUC were similar between the two formulations for total ketones, BHB, AcAc, tricaprilin, and octanoic acid. The variation was generally higher for tricaprilin than for the other analytes for both formulations.

The difference in PK of each analyte was also analyzed by cohort. The level of total ketones, BHB, AcAc, tricaprilin, and octanoic acid was generally higher in Chinese subjects than in Caucasian subjects when administered with the AC-SD-03 formulation, as measured by Cₘₐₓ and AUC, and the Tₘₐₓ was slightly longer to reach. Following AC-1202 formulation administration, the level of total ketones, BHB, AcAc, and octanoic acid was higher is Chinese subjects than in Caucasian subject as measured by the Cₘₐₓ and AUC, but the Tₘₐₓ was similar (except for AcAc that seems to have a longer Tₘₐₓ in Chinese than in Caucasian subjects). The overall level of tricaprilin was also generally higher in Chinese subjects than in Caucasian subjects as measured by the AUC, but a lower Cₘₐₓ was reached.

When administered the AC-SD-03 formulation, the variation in PK parameters was higher in Caucasian subjects than in Chinese subjects for total ketones, BHB, and AcAc. There were no obvious differences in the variation in PK parameters for octanoic acid. The PK parameters measured for tricaprilin following administration of this formulation were highly variable. The variation in Cₘₐₓ was 36% in Chinese compared to 71% in Caucasian subjects and the variation in AUC was 57% in Chinese compared to 34-43% in Caucasian subjects.

When administered the AC-1202 formulations, there were no obvious differences in variation in PK parameters between Chinese and Caucasian subjects for total ketones, BHB, AcAc, tricaprilin, and octanoic acid. The variation in Cₘₐₓ was generally higher for tricaprilin than for the other analytes for this formulation (Chinese: 63%, Caucasian: 50%). Also, for tricaprilin, the variation in AUC was significantly higher in Caucasian (60%) than in Chinese (25%) subjects.

Conclusions:

These results indicate that both tricaprilin formulations (AC-SD-03 and AC-1202) were well tolerated, apart from expected mild gastrointestinal symptoms, in healthy male volunteers, with no major safety concerns.

The production of total ketones, a critical pharmacodynamic marker for tricaprilin, was increased when the AC-1202 formulation was administered compared to the AC-SD-03 formulation. Although the exposure in Chinese men was numerically greater than in Caucasian men, the inter-individual variability within each cohort prevents a definitive conclusion that there is a metabolic difference between these ethnic groups.

### Example 4—PK Study in Healthy Older Subjects

A Phase 1, Single-centre, Multiple-dose, Open-label study to assess the Safety, Tolerability, and Pharmacokinetics of the AC-SD-03 Formulation of Tricaprilin in Healthy Older Volunteers

Objectives:

Primary Objective

To assess the safety and tolerability of multiple-dose administrations of tricaprilin formulated as AC-SD-03 administered using a titration scheme in healthy older volunteers.

Secondary Objective

To determine ketone body levels (total ketones, β-hydroxybutyrate [βHB], acetoacetate [AcAc]), after multiple-dose administrations of AC-SD-03 in healthy older volunteers.

Endpoints:

Primary Endpoint

Safety and tolerability outcomes were based on electrocardiogram (ECG) reports, gastrointestinal (GI) scales, vital sign measurements, clinical laboratory tests, adverse event (AE) reporting, and physical examination.

AEs and GI scales were tabulated and summary statistics for ECG, vital signs, and clinical laboratory safety tests may have been computed and provided, as deemed clinically appropriate.

Secondary Endpoint

Pharmacokinetic (PK) parameters (Cₘₐₓ, Tₘₐₓ, AUC0-4, AUC4-8, AUC0-8, and AUC0-24) were to be calculated for total ketones, βHB, and AcAc for the Day 27 24-hour PK sampling. Cₘₐₓ and Tₘₐₓ were calculated for total ketones, βHB, and AcAc for the Day 15 and Day 21 PK sampling.

Exploratory Endpoint

Potential effects of AC-SD-03 on liver outcomes were based on FibroScan reports, aspartate aminotransferase (AST):alanine aminotransferase (ALT) ratio.

Methodology:

This was an open-label, multiple-dose study to evaluate safety, tolerability, and limited PK of AC-SD-03, following titration to 75 g twice a day AC-SD-03 (30 g twice a day tricaprilin). The population for this study was 12 healthy older males and females age 50 and above.

Following a Screening period of up to 28 days, eligible subjects arrived at the clinical research unit (CRU) for Check-in on Day −1. On Day 1, subjects had predose samples of plasma serum taken for PK. Dose 1 of AC-SD-03 (12.5 g) was administered 30 minutes after completion of a standard breakfast. Dose 2 was administered 30 minutes after completion of a standard lunch.

Subjects were to gradually increase the dose according to the titration scheme, aiming to reach a dose of 75 g twice a day.

Titration Schedule of AC-SD-03

| | Dose 1 | | Dose 2 | |
|---|---|---|---|---|
| | | Containing | | Containing |
| Day | AC-SD-03 (g) | tricaprilin (g) | AC-SD-03 (g) | tricaprilin (g) |
| 1-4 | 12.5 | 5 | 12.5 | 5 |
| 5-9 | 25 | 10 | 25 | 10 |
| 10-15 | 37.5 | 15 | 37.5 | 15 |
| 16-21 | 50 | 20 | 50 | 20 |
| 22-28 | 75 | 30 | 75 | 30 |

In the event that the subject could not reach the target dose of 75 g twice a day over the course of the 4-week period, the subject would have reduced to the next highest tolerated dose and continued on this dose. At the discretion of the Investigator, a second attempt to escalate the dose once symptoms had settled may have been attempted. After a second failed attempt, the subject continued on the highest tolerated dose for the remainder of the study. However, subjects tolerated the dose titration as scheduled and no subject's regimen was modified for tolerability.

Subjects were to be confined from Day −1 until Day 28 but the subjects were released early due to the Covid-19 pandemic. Therefore, the final PK sample was drawn early and End-of-Study results were summarized as applicable for clinical laboratories, vital signs, and ECGs. On Days 15 and 21, subjects had predose and postdose (1, 1.5, and 2 hours) samples of plasma taken for PK. On Day 24, subjects had predose samples of plasma taken for PK. After the first dose on Day 24, 24-hour PK sampling was done to measure ketone body levels (βHB, AcAc). Check-out was on Day 25 following completion of scheduled assessments. Subjects who discontinued early from the study may have been replaced at the Sponsor's option.

A total of 12 subjects were enrolled in the study. Eleven (11) subjects completed treatment as per protocol but were withdrawn when the study was interrupted on Day 25 due to Covid-19 disruption and 1 subject withdrew on Day 23. All 12 subjects were included in safety and PK analyses. All subjects enrolled in this study were judged by the Investigator to be normal, healthy volunteers who met all inclusion and none of the exclusion criteria.

The test product was AC-SD-03 (tricaprilin oral powder for reconstitution), Lot No. A222000035. AC-SD-03 was weighed, mixed with 240 mL of water, shaken using a dosing container (with lid), and administered orally. Immediately after dosing, the remaining treatment in the container was rinsed with 60 mL of water and administered to the subject for a total of approximately 300 mL of dosing liquid consumed for each dosing. The total duration of participation, including the Screening period, for each subject was approximately 60 days.

Criteria for Evaluation:

Pharmacokinetics:

PK was evaluated based on PK parameters (Cₘₐₓ, Tₘₐₓ, AUC0-4, AUC4-8, AUC0-8, and AUC0-24) calculated for total ketones, βHB, and AcAc for the Day 24, 24-hour PK sampling. Cₘₐₓ and Tₘₐₓ were calculated for total ketones, βHB, and AcAc for the Day 15 and Day 21 PK sampling.

Safety:

Safety was evaluated based on 12-lead ECG reports, GI scales, vital sign measurements, clinical laboratory tests, AE reporting, and physical examination.

Statistical Methods:

Pharmacokinetics:

The plasma concentrations of βHB, AcAc, and total ketones were listed and summarized by study day and time point for all subjects in the PK Population. Mean and individual concentration-time profiles for Days 15, 21, and 24 were presented on linear and semi-log scales. Linear mean plots were presented with and without SD. Plasma βHB, AcAc and total ketones PK parameters were listed and summarized by study day and dose for all subjects in the PK Population. No inferential statistics were performed on the PK data.

Safety:

No inferential statistics were performed on safety data. Applicable continuous variables were summarized using sample size (n), arithmetic mean (Mean), standard deviation (SD), minimum, median, and maximum. Frequency counts and percentages were reported for categorical data when appropriate.

Results

Safety and Tolerability:

There were no deaths, serious adverse events (SAEs), or subject discontinuations due to AEs reported in the study. The AC-SD-03 formulation was well tolerated, and all subjects were able to titrate to the highest dose of 30 g tricaprilin BID. With reference to the table below, the most common AEs were gastrointestinal in nature, were mild, resolved and occurred mainly at the highest dose.

| | AC-SD-03 Dose Level | | | | |
|---|---|---|---|---|---|
| Adverse Event* | 12.5 g | 25 g | 37.5 g | 50 g | 75 g |
| Number of Subjects Dosed | 12(100%) | 12(100%) | 12(100%) | 12(100%) | 12(100%) |
| Number of Subjects With TEAEs | 3(25%) | 2(17%) | 4(33%) | 1(8%) | 6(50%) |
| Number of Subjects Without TEAEs | 9(75%) | 10(83%) | 8(67%) | 11(92%) | 6(50%) |
| Eye disorders | 0(0%) | 0(0%) | 1(8%) | 0(0%) | 0(0%) |
| Ocular hyperaemia | 0(0%) | 0(0%) | 1(8%) | 0(0%) | 0(0%) |
| Gastrointestinal disorders | 2(17%) | 2(17%) | 3(25%) | 0(0%) | 6(50%) |
| Abdominal discomfort | 0(0%) | 0(0%) | 0(0%) | 0(0%) | 2(17%) |
| Abdominal pain lower | 0(0%) | 1(8%) | 0(0%) | 0(0%) | 0(0%) |
| Abdominal pain upper | 1(8%) | 0(0%) | 1(8%) | 0(0%) | 2(17%) |
| Constipation | 1(8%) | 1(8%) | 1(8%) | 0(0%) | 3(25%) |
| Dyschezia | 0(0%) | 1(8%) | 0(0%) | 0(0%) | 0(0%) |
| Eructation | 0(0%) | 0(0%) | 0(0%) | 0(0%) | 1(8%) |
| Flatulence | 0(0%) | 0(0%) | 0(0%) | 0(0%) | 1(8%) |
| Haematochezia | 0(0%) | 1(8%) | 1(8%) | 0(0%) | 0(0%) |
| Nausea | 0(0%) | 0(0%) | 1(8%) | 0(0%) | 2(17%) |
| Salivary hypersecretion | 0(0%) | 0(0%) | 0(0%) | 0(0%) | 1(8%) |
| General disorders and | 0(0%) | 0(0%) | 1(8%) | 0(0%) | 1(8%) |
| administration site conditions | | | | | |
| Fatigue | 0(0%) | 0(0%) | 1(8%) | 0(0%) | 0(0%) |
| Feeling cold | 0(0%) | 0(0%) | 0(0%) | 0(0%) | 1(8%) |
| Immune system disorders | 1(8%) | 0(0%) | 0(0%) | 0(0%) | 0(0%) |
| Hypersensitivity | 1(8%) | 0(0%) | 0(0%) | 0(0%) | 0(0%) |
| Injury, poisoning and procedural | 0(0%) | 1(8%) | 0(0%) | 0(0%) | 0(0%) |
| complications | | | | | |
| Skin laceration | 0(0%) | 1(8%) | 0(0%) | 0(0%) | 0(0%) |
| Musculoskeletal and connective | 0(0%) | 0(0%) | 0(0%) | 0(0%) | 1(8%) |
| tissue disorders | | | | | |
| Back pain | 0(0%) | 0(0%) | 0(0%) | 0(0%) | 1(8%) |
| Nervous system disorders | 0(0%) | 0(0%) | 2(17%) | 1(8%) | 0(0%) |
| Headache | 0(0%) | 0(0%) | 2(17%) | 1(8%) | 0(0%) |
| Somnolence | 0(0%) | 0(0%) | 1(8%) | 0(0%) | 0(0%) |

Overall, a total 32 TEAEs were reported by 8 (67%) subjects in the study, with no trend in AE incidence noted in relation to AC-SD-03 dose level. Gastrointestinal AEs were commonly reported (67% of subject) in the study. The most common GI events included constipation, upper abdominal pain, and nausea. Overall, the majority of AEs reported in the study were mild in severity and considered at least possibly related to study drug. All AEs resolved by study completion. The majority of BARF and pain NRS scores were 0, indicative of no pain or abdominal discomfort. Low grade pain and/or discomfort was occasionally reported, with the majority of events reported at doses of 37.5 g AC-SD-03 and above. There were no treatment-related trends noted in the vital sign, clinical laboratory (including ALT/AST ratio) results, physical examination assessment, FibroScan, or safety ECG data in this study.

Pharmacokinetics:

The summary of plasma βHB, AcAc, and total ketones PK parameters are presented in the tables below:

### Summary of the Plasma BNB Unadjusted Pharmacokinetic Parameters Following Titration Over 4 Weeks to 75 g Twice a Day AC-SD-03 (30 g Twice a Day Tricaprilin) (PK Population)

| | Day 15/15 g | Day 21/20 g | Day 24/30 g |
|---|---|---|---|
| Pharmacokinetic | BID | BID | BID |
| Parameters | [n = 12] | [n = 12] | [n = 11] |
| Cₘₐₓ (μM) | 284 (40.5) | 455 (41.3) | 813 (30.2) |
| Tₘₐₓ (hr) | 1.49 (0.99, | 1.25 (1.00, | 6.50 (2.00, |
| | 1.99) | 2.08) | 7.01) |
| AUC0-4 (μM*hr) | NC | NC | 1710 (33.9) |
| AUC4-8 (μM*hr) | NC | NC | 1850 (29.0) |
| AUC0-8 (μM*hr) | NC | NC | 3560 (30.3) |
| AUC0-24 (μM*hr) | NC | NC | 4740 (32.2) |

Subjects received twice daily 12.5 g AC-SD-03 (5 g tricaprilin) on Days 1-4, 25 g AC-SD-03 (10 g tricaprilin) on Days 5-9, 37.5 g AC SD-03 (15 g tricaprilin) on Days 10-15, 50 g AC-SD-03 (20 g tricaprilin) on Days 16-21, and 75 g AC-SD-03 (30 g tricaprilin) on Days 22-24.
AUCs and Cₘₐₓ values are presented as geometric mean (geometric CV %). Tₘₐₓ values are presented as median (min, max).

### Summary of the Plasma AcAc Unadjusted Pharmacokinetic Parameters Following Titration Over 4 Weeks to 75 g Twice a Day AC-SD-03 (30 g Twice a Day Tricaprilin) (PK Population)

| | Day 15/15 g | Day 21/20 g | Day 24/30 g |
|---|---|---|---|
| Pharmacokinetic | BID | BID | BID |
| Parameters | [n = 12] | [n = 12] | [n = 11] |
| Cₘₐₓ (μM) | 96.6 (30.1) | 142 (38.4) | 224 (31.3) |
| Tₘₐₓ (hr) | 1.49 (0.99, | 1.25 (1.00, | 6.48 (1.20, |
| | 2.11) | 2.08) | 8.00) |
| AUC0-4 (μM*hr) | NC | NC | 508 (36.5) |
| AUC4-8 (μM*hr) | NC | NC | 577 (32.4) |
| AUC0-8 (μM*hr) | NC | NC | 1090 (34.1) |
| AUC0-24 (μM*hr) | NC | NC | 1700 (33.3) |

Subjects received twice daily 12.5 g AC-SD-03 (5 g tricaprilin) on Days 1-4, 25 g AC-SD-03 (10 g tricaprilin) on Days 5-9, 37.5 g AC SD-03 (15 g tricaprilin) on Days 10-15, 50 g AC-SD-03 (20 g tricaprilin) on Days 16-21, and 75 g AC-SD-03 (30 g tricaprilin) on Days 22-24.
AUCs and Cₘₐₓ values are presented as geometric mean (geometric CV %). Tₘₐₓ values are presented as median (min, max).

### Summary of the Plasma Total Ketones Unadjusted Pharmacokinetic Parameters Following Titration Over 4 Weeks to 75 g Twice a Day AC-SD-03 (30 g Twice a Day Tricaprilin) (PK Population)

| | Day 15/15 g | Day 21/20 g | Day 24/30 g |
|---|---|---|---|
| Pharmacokinetic | BID | BID | BID |
| Parameters | [n = 12] | [n = 12] | [n = 11] |
| Cₘₐₓ (μM) | 383 (35.7) | 599 (39.8) | 1040 (29.7) |
| Tₘₐₓ (hr) | 1.49 (0.99, | 1.25 (1.00, | 6.50 (2.00, |
| | 1.99) | 2.08) | 7.01) |
| AUC0-4 (μM*hr) | NC | NC | 2220 (33.6) |
| AUC4-8 (μM*hr) | NC | NC | 2430 (29.2) |
| AUC0-8 (μM*hr) | NC | NC | 4650 (30.6) |
| AUC0-24 (μM*hr) | NC | NC | 6440 (31.9) |

Subjects received twice daily 12.5 g AC-SD-03 (5 g tricaprilin) on Days 1-4, 25 g AC-SD-03 (10 g tricaprilin) on Days 5-9, 37.5 g AC SD-03 (15 g tricaprilin) on Days 10-15, 50 g AC-SD-03 (20 g tricaprilin) on Days 16-21, and 75 g AC-SD-03 (30 g tricaprilin) on Days 22-24.
AUCs and Cₘₐₓ values are presented as geometric mean (geometric CV %). Tₘₐₓ values are presented as median (min, max).

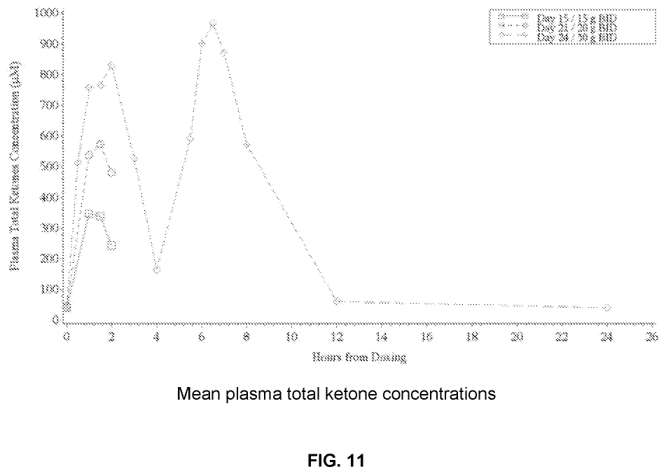

Following titration to 75 g twice a day AC-SD-03 (30 g tricaprilin) maximum concentrations of surrogate PK markers βHB, AcAc, and total ketones (FIG. 11) were observed between approximately 1 and 1.5 hours postdose on Days 15 and 21. On Day 24, when both dosing occasions were captured during the sampling period, maximum concentrations were observed at approximately 1.5 hours following the second dose administration. As expected, βHB was the most abundant. With reference to FIG. 11, mean plasma total ketone concentrations are shown over the titration period, at Day 15/15 g BID, Day 21/20 g BID, and Day 24/30 g BID.

Following titration from Day 15 (15 g tricaprilin) to Day 24 (30 g tricaprilin), geometric mean Cₘₐₓ for βHB, AcAc, and total ketones increased 2.9 fold, 2.3-fold, and 2.7-fold, respectively. On Day 24, overall exposure following the first dose (AUC0-4) was similar to that following the second dose based on the similar time interval (AUC4-8).

Concentrations following the first dose of the day did not return to baseline levels before the second dose of the day was administered. It is unlikely that endogenous ketosis occurred during the timeframe between meals, therefore, the concentration levels were likely due to administration of tricaprilin. Following the second dose, concentrations returned to baseline levels by approximately 12 hours.

Conclusions:

During the titration period, Tₘₐₓ of PK markers βHB, AcAc, and total ketones were observed between approximately 1 and 1.5 hours postdose on Days 15 and 21. Following titration to maximum dose, 75 g twice a day, on Day 24 Tₘₐₓ was 1.5 hours after second dose.

Following titration from Day 15 (15 g tricaprilin) to Day 24 (30 g tricaprilin), geometric mean Cₘₐₓ for βHB, AcAc, and total ketones increased 2.9-fold, 2.3-fold, and 2.7-fold, respectively. On Day 24, overall exposure following the first dose (AUC0-4) was similar to that following the second dose based on the similar time interval (AUC4-8). Following the second dose, concentrations returned to baseline levels by 12 hours.

Based on visual assessment of the curves, ketosis (as defined by levels of total ketones above 300 μM), was present for most of the daytime hours (up to 12 hours post-first meal of the day).

Multiple-dose administrations of tricaprilin formulated as AC-SD-03 administered using a titration scheme, with doses ranging from 12.5 g to 75 g AC-SD-03 (5 g to 30 g tricaprilin), appeared to be generally safe and well tolerated in the healthy older volunteers in this study. All subjects completed the titration schedule up to the maximal dose, and side effects were generally mild in severity and mostly GI related.

### Example 5—Ethnicity Analysis of Safety and Tolerability

The present example investigates the pharmacokinetics, safety and tolerability of tricaprilin in healthy young male Caucasian and Asian volunteers to understand and elucidate any differences between the two populations and any identify any ethnic sensitivities. In this example, data from several studies were analyzed to evaluate any ethnicity differences in safety and tolerability of tricaprilin. The analyzed studies included Caucasian and Asian (Chinese) subjects and several analyses were conducted to compare the effects in Caucasians vs Asian. Ethnic Chinese participants were defined as all four grandparents being Chinese. The level of total ketones were quantitated using a validated LC-MS/MS bioanalytical assay.

Methods: Study 1 was a food effect study of a spray dried formulation of tricaprilin (AC-SD-01), conducted in healthy young males. Study 2 was a 2-part study conducted in healthy young male volunteers, which tested a prototype, slow release spray dried formulation of tricaprilin (AC-SD-03); an earlier formulation of tricaprilin (AC-1202); and a placebo to AC-SD-03. Both of these studies included Caucasian and Asian (Chinese) subjects and several analyses were conducted to compare the effects in Caucasians vs Chinese. To explore whether ethnicity affects total ketone body exposure after tricaprilin administration, the pharmacokinetic parameters AUC₀₋ₜ and Cₘₐₓ from Study 2 were examined and grouped by an individual's ethnicity (Chinese or Caucasian).

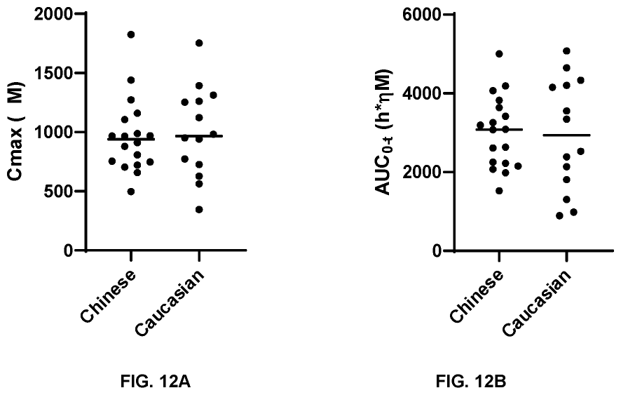

Results: Pharmacokinetic differences between ethnicities in each study were minor and were less apparent when corrected for weight. When data from the 2 parts of Study 2 were combined, the mean Cₘₐₓ for total ketones in Chinese participants was 965 uM and 1000 uM for Caucasian participants (p=0.78) and the mean total ketone AUC₀₋ₜ for Chinese participants was 3011 h*uM; whereas, for Caucasian participants, the AUC0-t was 2953 h*uM. (p=0.89). (See FIGS. 12A-12B. No differences were seen in AE profile between Asian and Caucasian subjects. In all studies, mild-moderate, self-limited GI adverse events were seen (bloating, nausea, abdominal discomfort). Further, based on a literature review, there are no known differences in the processes involved in the absorption, metabolism, distribution and elimination of medium chain triglycerides (MCTs) between Caucasians and Chinese, or in terms of the oxidation of medium chain fatty acids to ketone bodies.

Conclusions: Exposure to total ketones, the active species after tricaprilin administration was no different for healthy ethnic Chinese participants compared to healthy Caucasians. There does not appear to be any ethnic difference in absorption or metabolism of tricaprilin to produce ketone bodies, or in their safety and tolerability profile.

What is claimed is:

1. A method of administering tricaprilin for the treatment of a disease or disorder in a subject in need thereof, the method comprising:
administering a pharmaceutical composition comprising a therapeutically effective amount of tricaprilin to the subject in need thereof, wherein the therapeutically effective amount of tricaprilin provides a maximum serum concentration (Cₘₐₓ) of total ketones of at least 300 μmol/L; and
wherein the therapeutically effective amount of tricaprilin is between 30 g and 80 g per day, administered as single or divided doses.

2. The method of claim 1, wherein the therapeutically effective amount of tricaprilin provides a Cₘₐₓ of tricaprilin of at least 500 ng/mL.

3. The method of claim 1, wherein the therapeutically effective amount of tricaprilin provides a maximum serum concentration (Cₘₐₓ) of total ketones within at least 1 hour after administration, at least 1.5 hours after administration, at least 2 hours after administration, at least 2.5 hours after administration, or at least 3 hours after administration.

4. The method of claim 1, wherein the Cₘₐₓ of total ketones at least 500 μmol/L, at least 750 μmol/L, or at least 1000 μmol/L.

5. The method of claim 1, wherein the subject in need thereof is an elderly subject.

6. The method of claim 5, wherein the elderly subject lacks the ApoE4 genotype.

7. The method of claim 1, wherein the therapeutically effective amount of tricaprilin provides a Cₘₐₓ of b-hydroxybutyrate (BHB) of at least 400 μmol/L, at least 450 μmol/L, or at least 500 μmol/L.

8. The method of claim 1, wherein the therapeutically effective amount of tricaprilin provides a Cₘₐₓ of acetoacetate (AcAc) of at least 50 umol/L, at least 60 umol/L, at least 70 umol/L, at least 80 umol/L, at least 90 umol/L, or at least 100 umol/L.

9. The method of claim 1, wherein the disease or disorder is a disease or disorder associated with reduced cognitive function.

10. The method of claim 9, wherein the disease or disorder associated with reduced cognitive function is selected from Alzheimer's Disease and Age-Associated memory impairment.

11. The method of claim 1, wherein the pharmaceutical composition is formed as an emulsion for administration.

12. The method of claim 1, wherein the therapeutically effective does of tricaprilin of between 30 g and 80 g per day is achieved by titrating up to the final therapeutically effective dosage.

13. The method of claim 12, wherein the titration is performed over 2 to 4 weeks, with adjustments in dosage of 5 g to 10 g of tricaprilin per week.

14. The method of claim 1, wherein the pharmaceutical composition is administered such that no ethnicity affects in total ketone Cₘₐₓ exposure after tricaprilin administration is observed in Caucasian versus Asian subjects.
